SEQUENCE LISTING (4 sequences)

SEQ ID NO: 1 - DNA, 21 bp, Artificial Sequence
  Description of Artificial Sequence Synthetic oligonucleotide
ccatctacagctttccggcgc

SEQ ID NO: 2 - DNA, 20 bp, Artificial Sequence
  Description of Artificial Sequence Synthetic oligonucleotide
ctctggggtggccttcagca

SEQ ID NO: 3 - DNA, 20 bp, Artificial Sequence
  Description of Artificial Sequence Synthetic oligonucleotide
cggctaccacatccaaggaa

SEQ ID NO: 4 - DNA, 18 bp, Artificial Sequence
  Description of Artificial Sequence Synthetic oligonucleotide
gctggaattaccgcggct

(19) United States
(12) Patent Application Publication
(10) Pub. No.: US 2012/0003270 A1
(43) Pub. Date: Jan. 5, 2012

(54) 6-SUBSTITUTED ISOFLAVONOID COMPOUNDS AND USES THEREOF

(76) Inventors: Alan James Husband, Prymont (AU); Michael James, Dulwich (AU); Naresh Kumar, Maroubra (AU)

(21) Appl. No.: 13/056,608

(22) PCT Filed: Jul. 30, 2009
(86) PCT No.: PCT/AU09/00973

§ 371 Date: Sep. 22, 2011

(30) Foreign Application Priority Data
Jul. 30, 2008 (AU) 2008903873

(51) Int. Cl. A61K 31/352; C07D 405/10; A61K 31/4025; C12N 5/071; A61P 35/02; A61P 9/00; A61P 37/02; A61P 39/06; C07D 311/58; A61P 35/00

(52) U.S. Cl. 424/278.1; 549/406; 548/525; 514/456; 514/422; 435/375

(57) ABSTRACT

The present invention relates to 6-substituted isoflavonoid compounds and compositions comprising same. The invention further relates to the use of 6-substituted isoflavonoid compounds for the treatment of various diseases and conditions.

### TECHNICAL FIELD

The present invention relates to 6-substituted isoflavonoid compounds and compositions comprising same. The invention further relates to the use of 6-substituted isoflavonoid compounds for the treatment of various diseases and conditions.

### BACKGROUND OF THE INVENTION

Currently, the most commonly used anti-inflammatory agents are non-steroidal anti-inflammatory drugs (NSAIDs). Whilst NSAIDs are effective at reducing inflammation, their use has always been limited by their gastrointestinal side effects, such as gastric ulceration, perforation and bleeding, as well as acute renal failure and hypertension. These shortcomings were met in part by the development of agents which selectively inhibited the inflammatory process driven by COX-2, but left the homeostatic functions managed by COX-1 unaffected—the COXIBs. The theory was that the prostaglandin PGE₂ produced in response to COX-1, and which provided gut protection remained, but the PGE₂ synthesised in response to COX-2 produced as part of the inflammatory response was suppressed.

However, the consequence of inhibiting COX-2 alone is that more PGH₂ is made available for the production of other COX-1-derived eicosanoids, in particular thromboxane (TXA₂) which causes platelet aggregation (Caughey et al. 2001). It has been established that there is an increase in adverse cardiovascular events associated with selective COX-2 inhibition. There is also now clear evidence that all NSAIDs are associated with some cardiovascular risk (Fosslien 2005).

These developments accentuate the shortcomings in currently available anti-inflammatory therapeutics. Regardless of the COX isotype inhibited and in what proportions, inhibiting inflammation via the COX pathway is accompanied by the complication of gastrointestinal, renal and cardiovascular side effects.

Various gut protective strategies have been employed, for example co-therapy with proton pump inhibitors or synthetic PGE₂ analogues such as misoprostol. ‘Safer’ agents like nitric oxide-donating NSAIDs (NO-NSAIDs) and NSAIDs coupled to a synthetic version of one of the phospholipids in the mucous layer of the stomach, phosphatidylcholine (PC-NSAIDs) are still in development.

Nevertheless, the underlying problem with existing agents, as well as any strategies to improve their safety profile, is that they all inhibit COX. The cardiovascular risks associated with selective COX-2 inhibition appear to be due to the disruption of the homeostasis between COX-2-induced prostacyclin (PGI₂), which is anti-thrombotic and vasodilatory, and COX-1-induced TXA₂, which is prothrombotic and vasoconstrictory (Caughey et al. 2001). Moreover, TXA₂ promotes and PGI₂ prevents the initiation and progression of atherogenesis through control of platelet activation and leukocyte-endothelial cell interaction (Kobayashi et al. 2004). This homeostasis is disturbed to varying degrees whenever the COX pathway is inhibited, regardless of COX isotype selectivity, as has been well demonstrated with the increased cardiovascular and gastrointestinal side effects associated with all NSAIDs.

It is clear that the enormous need for safe anti-inflammatory agents remains unchanged. The regulatory developments regarding safety issues and labelling of NSAIDs and COXIBs make the need for new anti-inflammatory agents even more critical. An ideal anti-inflammatory therapeutic would possess anti-inflammatory activity without the cardiovascular risks caused by the inhibition of COX.

The present inventors have surprisingly found that certain 6-substituted isoflavonoid compounds possess useful anti-inflammatory activity. In addition, it has been discovered that certain 6-substituted isoflavanoid compounds may also provide other therapeutic benefits.

### SUMMARY OF THE INVENTION

In a first aspect, the present invention provides a compound of the general formula (I):

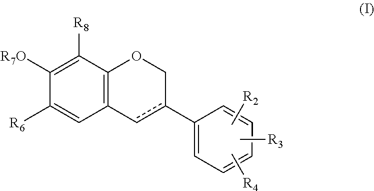

wherein:

R₂, R₃ and R₄ are independently selected from the group consisting of: hydrogen, hydroxy, OR₉, OC(O)R₉, OSi(R₁₀)₃, C₁-C₁₀alkyl, C₃-C₇ cycloalkyl, amino, aminoalkyl, aryl, arylalkyl, alkylaryl, thiol, COOH, alkylthio, nitro, cyano, halo, C₂-C₆ alkenyl, C₂-C₆ alkynyl and heteroaryl,

R₆ is R₁₁(R₁₂)N(CH₂)ₙ—,

R₇ is selected from the group consisting of: hydrogen, R₉, C(O)R₉, Si(R₁₀)₃ and C₃-C₇ cycloalkyl,

R₈ is selected from the group consisting of: hydrogen, C₁-C₁₀, alkyl, C₃-C₇ cycloalkyl, aryl, arylalkyl, nitro, cyano and halo,

R₉ is selected from the group consisting of: C₁-C₁₀alkyl, haloalkyl, aryl, arylalkyl and alkylaryl,

R₁₀ is independently selected from the group consisting of: C₁-C₁₀ alkyl and aryl,

R₁₁ and R₁₂ are independently selected from the group consisting of: hydrogen, C₁-C₁₀ alkyl and —Y—CO₂R₁₃, or R₁₁ and R₁₂ together with the nitrogen to which they are attached form a heterocyclic ring comprising 5, 6 or 7 ring members, the heterocyclic ring being optionally substituted with one or more substituents selected from the group consisting of: C₁-C₁₀ alkyl, C₂-C₆ alkenyl, C₂-C₆ alkynyl, COOH, COOR₁₀, halo, nitro, cyano and aryl,

R₁₃ is selected from the group consisting of: hydrogen, C₃-C₇ cycloalkyl, C₁-C₁₀ alkyl, C₂-C₆-alkenyl and C₂-C₆ alkynyl,

Y is a hydrocarbon chain having between 1 and 15 carbon atoms which may optionally be interrupted by one or more oxygen, nitrogen or sulfur atoms,

n is an integer between 1 and 4,

the drawing [symbol] represents either a single bond or a double bond, and salts thereof.

The compound of the formula (I) may be selected from the group consisting of:

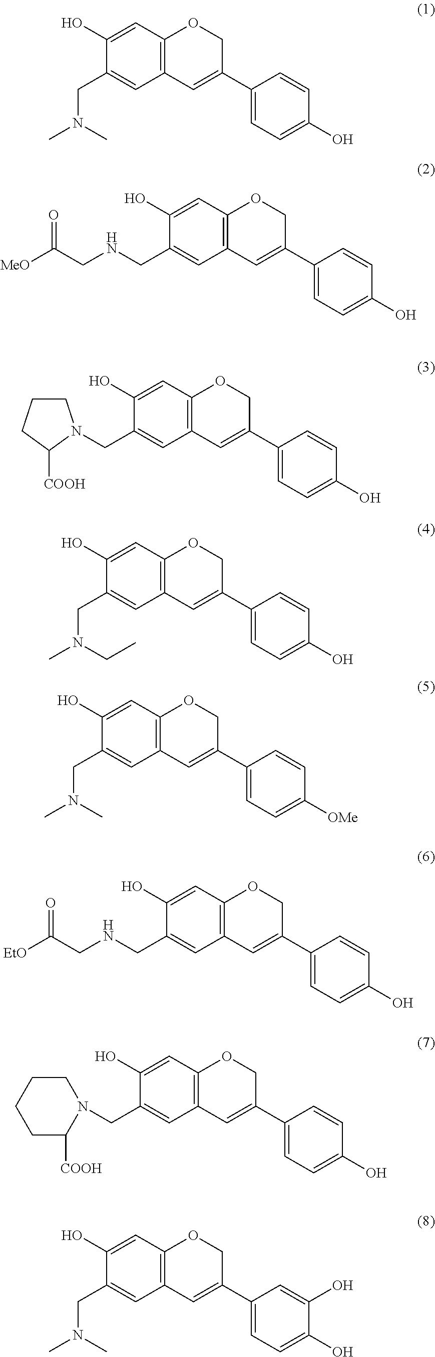

In a second aspect, the present invention provides a pharmaceutical composition comprising a compound of the formula (I), or a pharmaceutically acceptable salt thereof, as defined in the first aspect, and a pharmaceutically acceptable carrier, diluent and/or excipient.

In a third aspect, the present invention provides a method for the prevention and/or treatment of inflammation and/or an inflammatory disease or disorder in a subject in need thereof, said method comprising administration to the subject of a therapeutically effective amount of a compound of the formula (I), or a pharmaceutically acceptable salt thereof, as defined in the first aspect.

In a fourth aspect, the present invention provides the use of a compound of the formula (I), or a pharmaceutically acceptable salt thereof, as defined in the first aspect in the manufacture of a medicament for the prevention and/or treatment of inflammation and/or an inflammatory disease or disorder.

In a fifth aspect, the present invention provides a compound of the formula (I), or a pharmaceutically acceptable salt thereof, as defined in the first aspect, for use in the prevention and/or treatment of inflammation and/or an inflammatory disease or disorder.

In a sixth aspect, the present invention provides the use of a compound of the formula (I) or a pharmaceutically acceptable salt thereof, as defined in the first aspect, as an antioxidant.

In a seventh aspect, the present invention provides a method for modulation of the immune system in a subject, said method comprising administration to the subject of a therapeutically effective amount of a compound of the formula (I), or a pharmaceutically acceptable salt thereof, as defined in the first aspect.

In an eighth aspect, the present invention provides the use of a compound of the formula (I), or a pharmaceutically acceptable salt thereof, as defined in the first aspect in the manufacture of a medicament for modulation of the immune system.

In a ninth aspect, the present invention provides the use of a compound of the formula (I) or a pharmaceutically acceptable salt thereof, as defined in the first aspect, for modulation of the immune system.

The modulation of the immune system may comprise inhibition or suppression of an immune response.

The modulation of the immune system may comprise suppression of activation or production of T-cells and/or B-cells.

In a tenth aspect, the present invention provides a method for inhibiting the proliferation of cells, said method comprising contacting the cells with a compound of the formula (I), or a pharmaceutically acceptable salt thereof, as defined in the first aspect.

In an eleventh aspect, the present invention provides the use of a compound of the formula (I), or a pharmaceutically acceptable salt thereof, as defined in the first aspect in the manufacture of a medicament for inhibiting the proliferation of cells.

In a twelfth aspect, the present invention provides a compound of the formula (I), or a pharmaceutically acceptable salt thereof, as defined in the first aspect, for use in inhibiting the proliferation of cells.

In a thirteenth aspect, the present invention provides a method for the prevention and/or treatment of cancer in a subject in need thereof, said method comprising administration to the subject of a therapeutically effective amount of a compound of the formula (I), or a pharmaceutically acceptable salt thereof, as defined in the first aspect.

In a fourteenth aspect, the present invention provides the use of a compound of the formula (I), or a pharmaceutically acceptable salt thereof, as defined in the first aspect in the manufacture of a medicament for the prevention and/or treatment of cancer.

In a fifteenth aspect, the present invention provides a compound of the formula (I), or a pharmaceutically acceptable salt thereof, as defined in the first aspect, for use in the prevention and/or treatment of cancer.

The cancer may be selected from the group consisting of: ovarian cancer, leukaemia, prostate cancer, colorectal cancer, pancreatic cancer, glioma, melanoma and lung cancer.

In a sixteenth aspect, the present invention provides a method for the prevention and/or treatment of cardiovascular disease in a subject in need thereof, said method comprising administration to the subject of a therapeutically effective amount of a compound of the formula (I), or a pharmaceutically acceptable salt thereof, as defined in the first aspect.

In a seventeenth aspect, the present invention provides the use of a compound of the formula (I), or a pharmaceutically acceptable salt thereof, as defined in the first aspect in the manufacture of a medicament for the prevention and/or treatment of cardiovascular disease.

In an eighteenth aspect, the present invention provides a compound of the formula (I), or a pharmaceutically acceptable salt thereof, as defined in the first aspect, for use in the prevention and/or treatment of cardiovascular disease.

### DEFINITIONS

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

In the context of this specification, the terms “treatment” and “treating” refer to any and all uses which remedy a condition, disease, disorder or symptoms thereof, or otherwise prevent, hinder or reverse the progression of a condition, disease, disorder or symptoms thereof, in any way whatsoever. Treatment may be for a defined period of time, or provided on an ongoing basis depending on the particular circumstances of any given individual.

In the context of this specification, the terms “prevent” and “prevention” refer to any and all uses which prevent the establishment or onset of a condition, disease, disorder or symptoms thereof in any way whatsoever.

In the context of this specification, the term “therapeutically effective amount” includes within its meaning a non-toxic amount of a compound of formula (I) sufficient to provide the desired therapeutic effect. The exact amount will vary from subject to subject depending on the age of the subject, their general health, the severity of the disorder being treated and the mode of administration. It is therefore not possible to specify an exact “therapeutically effective amount”, however one skilled in the art would be capable of determining a “therapeutically effective amount” by routine trial and experimentation.

In the context of this specification, the term “salts thereof” is understood to include acid addition salts, anionic salts and zwitterionic salts, and in particular pharmaceutically acceptable salts.

In the context of this specification, “pharmaceutically acceptable salts” include, but are not limited to, those formed from: acetic, ascorbic, aspartic, benzoic, benzenesulfonic, citric, cinnamic, ethanesulfonic, fumaric, glutamic, glutaric, gluconic, hydrochloric, hydrobromic, lactic, maleic, malic, methanesulfonic, naphthoic, hydroxynaphthoic, naphthalenesulfonic, naphthalenedisulfonic, naphthaleneacrylic, oleic, oxalic, oxaloacetic, phosphoric, pyruvic, p-toluenesulfonic, tartaric, trifluoroacetic, triphenylacetic, tricarballylic, salicylic, sulphuric, sulfamic, sulfanilic and succinic acid.

In the context of this specification, the term “C₁-C₁₀ alkyl” is taken to include straight chain and branched chain monovalent saturated hydrocarbon groups having 1 to 10 carbon atoms, such as methyl, ethyl, propyl, isopropyl, butyl, isobutyl, secbutyl, tertiary butyl, pentyl, hexyl, heptyl, octyl, nonyl, decyl and the like.

In the context of this specification, the term “C₁-C₆ alkyl” is taken to include straight chain and branched chain monovalent saturated hydrocarbon groups having 1 to 6 carbon atoms, such as methyl, ethyl, propyl, isopropyl, butyl, isobutyl, secbutyl, tertiary butyl, pentyl, hexyl and the like.

In the context of this specification, the term “C₂-C₆ alkenyl” is taken to include straight chain and branched chain monovalent hydrocarbon radicals having 2 to 6 carbon atoms and at least one carbon-carbon double bond, such as vinyl, propenyl, 2-methyl-2-propenyl, butenyl, pentenyl and the like. The alkenyl group may contain from 2 to 4 carbon atoms.

In the context of this specification, the term “C₂-C₆ alkynyl” is taken to include straight chain and branched chain monovalent hydrocarbon radicals having 2 to 6 carbon atoms and at least one carbon-carbon triple bond, such as ethynyl, propynyl, butynyl, pentynyl, hexynyl and the like. The alkynyl group may contain from 2 to 4 carbon atoms.

In the context of this specification, the term “C₃-C₇ cycloalkyl” is taken to include cyclic alkyl groups having 3 to 7 carbon atoms such as cyclopropyl, cyclobutyl, cyclopentyl, cyclohexyl and cycloheptyl.

The alkyl, alkenyl, alkynyl or cycloalkyl group may optionally be substituted by one or more of: acyloxy, hydroxy, halo, alkoxy, nitro or cyano.

In the context of this specification, the term “aryl” is taken to include monovalent aromatic radicals having between 6 and 30 carbon atoms. The aryl group may be selected from the group consisting of: phenyl, biphenyl, naphthyl, anthracenyl and phenanthrenyl. The aryl group may be unsubstituted or optionally substituted by one or more of: C₁-C₆ alkyl, halo, acyloxy, hydroxy, alkoxy, silyloxy, nitro or cyano.

In the context of this specification, the term “heteroaryl” is taken to include monovalent aromatic radicals having between 1 and 12 atoms, wherein 1 to 6, or 1 to 5, or 1 to 4, or 1 to 3, or 1 or 2 atoms are heteroatoms selected from nitrogen, oxygen and sulfur. The heteroaryl group may be selected from the group consisting of: furanyl, quinazolinyl, quinolinyl, isoquinolinyl, indolyl, benzimidazolyl, pyrazolyl, tetrazolyl, oxazolyl, isoxazolyl, isothiazolyl, thiazolyl, thienyl, imidazolyl, pyrazinyl, pyridazinyl, pyrimidinyl, pyridyl, triazolyl, benzothiazolyl, benzisothiazolyl, benzoxazolyl, benzisoxazolyl, benzimidazolyl and triazinyl. The heteroaryl group may be unsubstituted or optionally substituted by one or more of: alkyl, halo, acyloxy, hydroxy, halo, alkoxy, silyloxy, nitro or cyano.

In the context of this specification, the term “halo” is taken to include fluoro, chloro, bromo and iodo.

In the context of this specification, the term “aminoalkyl” is taken to include “alkyl” as defined above, wherein one or more hydrogen atoms have been replaced by one or more amino groups. One or two hydrogen atoms may be replaced by one or two amino groups. The aminoalkyl group may be aminomethyl, aminoethyl, aminopropyl and the like.

In the context of this specification, the term “arylalkyl” is taken to include an “aryl” group as defined above attached to the molecule via a divalent alkylene group. Examples of arylalkyl groups include benzyl and phenethyl and the like. The term “alkylene” is taken to include a divalent group derived from a straight or branched chain saturated hydrocarbon group by the removal of two hydrogen atoms. Representative alkylene groups include methylene, ethylene, propylene, isobutylene, and the like.

In the context of this specification, the term “alkylaryl” is taken to include an “alkyl” group as defined above attached to the molecule via a divalent arylene group. Examples of alkylaryl groups include tolyl, ethylphenyl, propylphenyl, butylphenyl and the like. The term “arylene” is taken to include an aromatic ring system derived from an aryl group as defined above by the removal of two hydrogen atoms.

In the context of this specification, the term “haloalkyl” is taken to include monohalogenated, dihalogenated and up to perhalogenated alkyl groups. Preferred perhaloalkyl groups are trifluoromethyl and pentafluoroethyl.

### BRIEF DESCRIPTION OF THE FIGURES

FIG. 1: Effect of compound (1) (denoted as NV-17) on joint scores in rat adjuvant-induced arthritis.

FIG. 2: Effect of incubation with compound (1) on splenocyte proliferation and cytokine production.

FIG. 3: Effect of incubation with compound (1) on splenocyte proliferation and cytokine production.

FIG. 4: Effect of compound (1) (denoted as NV-17) on the aortic contractility induced by noradrenaline.

FIG. 5: Effect of compound (3) (denoted as NV-124) on the aortic contractility induced by noradrenaline.

### DETAILED DESCRIPTION OF THE INVENTION

In a first aspect, the present invention provides a compound of the general formula (I):

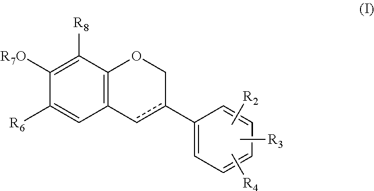

wherein:

R₂, R₃ and R₄ are independently selected from the group consisting of: hydrogen, hydroxy, OR₉, OC(O)R₉, OSi(R₁₀)₃, C₁-C₁₀ alkyl, C₃-C₇ cycloalkyl, amino, aminoalkyl, aryl, arylalkyl, alkylaryl, thiol, COOH, alkylthio, nitro, cyano, halo, C₂-C₆ alkenyl, C₂-C₆ alkynyl and heteroaryl,

R₆ is R₁₁(R₁₂)N(CH₂)ₙ—,

R₇ is selected from the group consisting of: hydrogen, R₉, C(O)R₉, Si(R₁₀)₃ and C₃-C₇ cycloalkyl,

R₈ is selected from the group consisting of: hydrogen, C₁-C₁₀ alkyl, C₃-C₇ cycloalkyl, aryl, arylalkyl, nitro, cyano and halo,

R₉ is selected from the group consisting of: C₁-C₁₀ alkyl, haloalkyl, aryl, arylalkyl and alkylaryl,

R₁₀ is independently selected from the group consisting of: C₁-C₁₀ alkyl and aryl,

R₁₁ and R₁₂ are independently selected from the group consisting of: hydrogen, C₁-C₁₀ alkyl and —Y—CO₂R₁₃, or R₁₁ and R₁₂ together with the nitrogen to which they are attached form a heterocyclic ring comprising 5, 6 or 7 ring members, the heterocyclic ring being optionally substituted with one or more substituents selected from the group consisting of: C₁-C₁₀ alkyl, C₂-C₆ alkenyl, C₂-C₆ alkynyl, COOH, COOR₁₀, halo, nitro, cyano and aryl,

R₁₃ is selected from the group consisting of: hydrogen, C₃-C₇ cycloalkyl, C₁-C₁₀ alkyl, C₂-C₆-alkenyl and C₂-C₆ alkynyl,

Y is a hydrocarbon chain having between 1 and 15 carbon atoms which may optionally be interrupted by one or more oxygen, nitrogen or sulfur atoms,

n is an integer between 1 and 4,

the drawing [symbol] represents either a single bond or a double bond, and salts thereof.

R₂, R₃ and R₄ may be independently selected from the group consisting of: hydrogen, C₁-C₁₀ alkyl, halo, hydroxy, OR₉, OC(O)R₉ and OSi(R₁₀)₃. In one embodiment, at least one of R₂, R₃ and R₄ is hydroxy. In an alternative embodiment, R₂, R₃ and R₄ are independently selected from the group consisting of: hydrogen and hydroxy, wherein at least two of R₂, R₃ and R₄ are hydrogen and the remaining substituent is hydroxy. The hydroxy substituent (when present) may be located at the para position.

R₇ may be selected from the group consisting of: hydrogen, C(O)R₉ and C₁-C₁₀ alkyl.

R₈ may be selected from the group consisting of: hydrogen, C₁-C₁₀ alkyl, aryl, arylalkyl and halo.

R₉ may be selected from the group consisting of: C₁-C₁₀ alkyl, haloalkyl and aryl.

R₁₀ may be C₁-C₁₀ alkyl.

R₁₁ and R₁₂ may be independently selected from the group consisting of: —Y—CO₂R₁₃, hydrogen and C₁-C₁₀ alkyl, or R₁₁ and R₁₂ together with the nitrogen to which they are attached form a heterocyclic ring comprising 5 or 6 ring members, the heterocyclic ring being optionally substituted with one or more substituents selected from the group consisting of: C₁-C₁₀ alkyl, COOH, COOR₁₀ and halo.

Y may be a hydrocarbon chain having between 1 and 10, 1 and 9, 1 and 8, 1 and 7 or 1 and 6 carbon atoms.

R₁₃ may be C₁-C₁₀ alkyl.

n may be 1, 2 or 3.

R₂, R₃ and R₄ may be independently selected from the group consisting of hydrogen, hydroxy and OR₉.

R₇ may be selected from the group consisting of: hydrogen and C₁-C₆ alkyl.

R₈ may be selected from the group consisting of: hydrogen, C₁-C₁₀ alkyl and halo.

R₉ may be selected from the group consisting of: C₁-C₆ alkyl and haloalkyl.

R₁₀ may be C₁-C₆ alkyl.

R₁₁ and R₁₂ may be independently selected from the group consisting of: —Y—CO₂R₁₃, hydrogen and C₁-C₆ alkyl, or R₁₁ and R₁₂ together with the nitrogen to which they are attached form a heterocyclic ring comprising 5 or 6 ring members, the heterocyclic ring being optionally substituted with one or more substituents selected from the group consisting of: C₁-C₁₀ alkyl, COOH and halo.

Y may be a hydrocarbon chain having between 1 and 5 carbons.

R₁₃ may be C₁-C₆ alkyl.

n may be 1 or 2.

R₂, R₃ and R₄ may be independently selected from the group consisting of: hydrogen, hydroxy and OMe.

R₇ may be selected from the group consisting of: hydrogen and methyl.

R₈ may be selected from the group consisting of: hydrogen and C₁-C₆ alkyl.

R₉ may be C₁-C₆ alkyl.

R₁₁ and R₁₂ may be independently selected from the group consisting of: —Y—CO₂R₁₃, hydrogen and methyl, or R₁₁ and R₁₂ together with the nitrogen to which they are attached form a heterocyclic ring comprising 5 ring members, the heterocyclic ring being optionally substituted with a substituent selected from the group consisting of: methyl, COOH and halo.

Y may be a hydrocarbon chain having 1 or 2 carbon atoms.

R₁₃ may be selected from the group consisting of methyl, ethyl or propyl.

n is 1.

In one embodiment, R₂, R₃ and R₄ are independently selected from the group consisting of: hydrogen, hydroxy, OR₉, OC(O)R₉ and OSi(R₁₀)₃, R₇ is selected from the group consisting of: hydrogen, C(O)R₉ and C₁-C₁₀ alkyl, R₈ is selected from the group consisting of: hydrogen, C₁-C₁₀ alkyl, aryl, arylalkyl, and halo, R₉ is selected from the group consisting of: C₁-C₁₀ alkyl, haloalkyl and aryl, R₁₀ is C₁-C₁₀ alkyl, R₁₁ and R₁₂ are independently selected from the group consisting of: —Y—CO₂R₁₃, hydrogen and C₁-C₁₀ alkyl, or R₁₁ and R₁₂ together with the nitrogen to which they are attached form a heterocyclic ring comprising 5 or 6 ring members, the heterocyclic ring being optionally substituted with one or more substituents selected from the group consisting of: C₁-C₁₀ alkyl, COOMe, COOH and halo, Y is a hydrocarbon chain having between 1 and 10 carbons, R₁₃ is C₁-C₁₀ alkyl and n is 1, 2 or 3.

In another embodiment, R₂, R₃ and R₄ are independently selected from the group consisting of: hydrogen and hydroxy, wherein at least one of R₂, R₃ and R₄ is hydroxy, R₇ is selected from the group consisting of: hydrogen and C₁-C₁₀ alkyl, R₈ is selected from the group consisting of: hydrogen, C₁-C₁₀ alkyl and halo, R₁₁ and R₁₂ are independently selected from the group consisting of: —Y—CO₂R₁₃, hydrogen and C₁-C₁₀ alkyl or R₁₁ and R₁₂ together with the nitrogen to which they are attached form a heterocyclic ring comprising 5 ring members, the heterocyclic ring being optionally substituted with one or more substituents selected from the group consisting of: C₁-C₁₀ alkyl, COOMe and COOH, Y is a hydrocarbon chain having between 1 and 6 carbons, R₁₃ is C₁-C₆ alkyl and n is 1 or 2.

In a further embodiment, R₂, R₃ and R₄ are independently selected from the group consisting of: hydrogen and hydroxy, wherein at least one of R₂, R₃ and R₄ is hydroxy, R₇ is selected from the group consisting of: hydrogen and C₁-C₆ alkyl, R₈ is selected from the group consisting of: hydrogen and C₁-C₆ alkyl, R₁₁ and R₁₂ are independently selected from the group consisting of: —Y—CO₂R₁₃, hydrogen and C₁-C₆ alkyl or R₁₁ and R₁₂ together with the nitrogen to which they are attached form a heterocyclic ring comprising 5 ring members, the heterocyclic ring being optionally substituted with one or more substituents selected from the group consisting of: C₁-C₆ alkyl, COOH and COOMe, Y is a hydrocarbon chain having between 1 and 4 carbons, R₁₃ is C₁-C₆ alkyl, n is 1 or 2 and the double bond at position 3 is present.

In another embodiment, R₂, R₃ and R₄ are independently selected from the group consisting of: hydrogen and hydroxy, wherein at least one of R₂, R₃ and R₄ is hydroxy, R₇ is hydrogen, R₈ is selected from the group consisting of: hydrogen and C₁-C₆ alkyl, R₁₁ and R₁₂ are independently selected from the group consisting of: —Y—CO₂R₁₃, hydrogen and C₁-C₆ alkyl or R₁₁ and R₁₂ together with the nitrogen to which they are attached form a heterocyclic ring comprising 5 ring members, the heterocyclic ring being optionally substituted with one or two substituents selected from the group consisting of: C₁-C₆ alkyl, COOH and COOMe, Y is a hydrocarbon chain having between 1 and 4 carbons, R₁₃ is C₁-C₆ alkyl, n is 1 or 2 and the double bond at position 3 is present.

In yet another embodiment, R₂, R₃ and R₄ are independently selected from the group consisting of: hydrogen and hydroxy, wherein at least one of R₂, R₃ and R₄ is hydroxy, R₇ is hydrogen, R₈ is hydrogen, R₁₁ and R₁₂ are independently selected from the group consisting of: —Y—CO₂R₁₃, hydrogen, methyl, ethyl, n-propyl, isopropyl, n-butyl, t-butyl and s-butyl or R₁₁ and R₁₂ together with the nitrogen to which they are attached form a heterocyclic ring comprising 5 ring members, the heterocyclic ring being optionally substituted with one or two substituents selected from the group consisting of: methyl, ethyl and COOH, Y is a hydrocarbon chain having between 1 and 3 carbons, R₁₃ is methyl, ethyl, isopropyl or propyl, n is 1 or 2 and the double bond at position 3 is present.

In still a further embodiment, R₂, R₃ and R₄ are independently selected from the group consisting of: hydrogen and hydroxy, wherein at least two of R₂, R₃ and R₄ are hydrogen and the remaining substituent is hydroxy, R₇ is hydrogen, R₈ is hydrogen, R₁₁ and R₁₂ are independently selected from the group consisting of: —Y—CO₂R₁₃, hydrogen, methyl, ethyl, n-propyl and isopropyl or R₁₁ and R₁₂ together with the nitrogen to which they are attached form a heterocyclic ring comprising 5 ring members, the heterocyclic ring being optionally substituted with COOH, Y is —CH₂— or —CH₂CH₂—, R₁₃ is methyl, ethyl, isopropyl or propyl, n is 1 or 2 and the double bond at position 3 is present.

In another embodiment, R₂, R₃ and R₄ are independently selected from the group consisting of: hydrogen and hydroxy, wherein at least two of R₂, R₃ and R₄ are hydrogen and the remaining substituent is hydroxy which is located in the para position, R₇ is hydrogen, R₈ is hydrogen, R₁₁ and R₁₂ are independently selected from the group consisting of: —Y—CO₂R₁₃, hydrogen, methyl, ethyl, n-propyl and isopropyl or R₁₁ and R₁₂ together with the nitrogen to which they are attached form a heterocyclic ring comprising 5 ring members, the heterocyclic ring being optionally substituted with COOH, Y is —CH₂— or —CH₂CH₂—, R₁₃ is methyl, ethyl, isopropyl or propyl, n is 1 or 2 and the double bond at position 3 is present.

In yet a further embodiment, R₂, R₃ and R₄ are independently selected from the group consisting of: hydrogen and hydroxy, wherein at least two of R₂, R₃ and R₄ are hydrogen and the remaining substituent is hydroxy which is located in the para position, R₇ is hydrogen, R₈ is hydrogen, R₁₁ and R₁₂ are independently selected from the group consisting of: —Y—CO₂R₁₃, hydrogen, methyl, ethyl, n-propyl and isopropyl, R₁₃ is methyl, ethyl, isopropyl or n-propyl, Y is —CH₂—, n is 1 and the double bond at position 3 is present.

In an embodiment of the invention, the compound of the formula (I) may be a compound wherein the pendant phenyl ring located at the 3-position of the benzopyran ring is less activated than the phenyl ring of the actual benzopyran moiety.

The compounds of formula (I) may have one or more chiral centres. The present invention includes all enantiomers and diastereoisomers, as well as mixtures thereof in any proportions. The invention also extends to isolated enantiomers or pairs of enantiomers. Enantiomers and diastereoisomers may be separated according to methods well known to those skilled in the art.

### Synthesis of Compounds of Formula (I)

Compounds of the formula (I) may be prepared from known starting materials according to Scheme 1 for example.

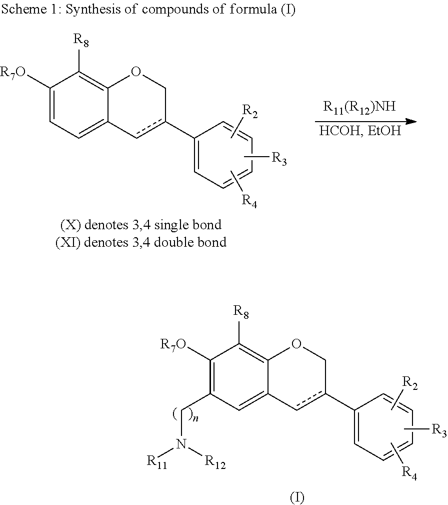

As shown in Scheme 1, a compound of the formula (X) or (X1) may be treated with an appropriately functionalised amino compound in the presence of formaldehyde to yield a compound of formula (I) having an amino-containing substituent at the 6-position. Those skilled in the art will realise that alternative synthetic routes may be employed in order to prepare compounds of the formula (I).

Compounds of the formula (X) and (X1) may be prepared according to standard methods, such as for example the method depicted in Scheme 2.

Access to various substitution patterns around the benzopyran ring and the pendant phenyl ring is possible by selecting correspondingly substituted R₇ and R₈ phenols (A) and R₂-R₄-phenyl acetic acid (B) starting materials according to, for example, published International application Nos. WO 98/08503 and WO 01/17986, and references cited therein, the disclosures of which are incorporated herein by reference.

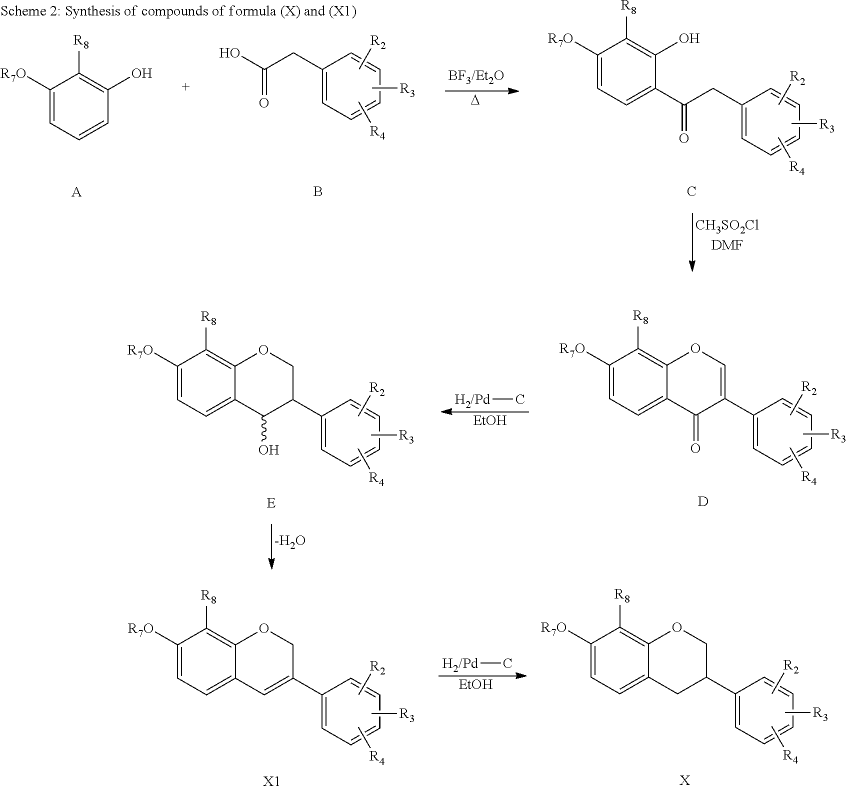

The ring cyclisation reactions of compounds of formula (C) may be conveniently performed with methanesulfonyl chloride to give compounds of formula (D). The reduction reaction to isoflavanols of formula (E) may be carried out with Pd—C or Pd-alumina in an alcoholic solvent in the presence of hydrogen. Dehydration may be effected with acid or P₂O₅ for example to afford compounds of the formula (X1).

The hydrogenation and dehydration reactions generally work better when hydroxy groups present are first protected. The protection of the hydroxy groups can be carried out by well established methods in the art, for example as described in T. W. Greene, “Protective Groups in Organic Synthesis”, John Wiley & Sons, New York, 1981. Hydroxy protecting groups include, carboxylic acid esters, e.g. acetate esters, aryl esters such as benzoate, acetals/ketals such as acetonide and benzylidene, ethers such as ortho-benzyl and methoxy benzyl ethers, tetrahydropyranyl ethers and silyl ethers such as tert-butyldimethyl silyl ether. Protecting groups can be removed by, for example, acid or base catalysed hydrolysis or reduction, for example, hydrogenation. Silyl ethers may require hydrogen fluoride or tetrabutylammonium fluoride to be cleaved.

Compounds of the formula (X1) may be further hydrogenated to provide compounds of the formula (X), if it is desired to prepare compounds of formula (I) wherein the optional double bond is not present.

Persons skilled in the art will be aware that other standard methods known to those skilled in the art may be used to prepare compounds of the formula (X) and (X1).

Compounds of the invention include the following:

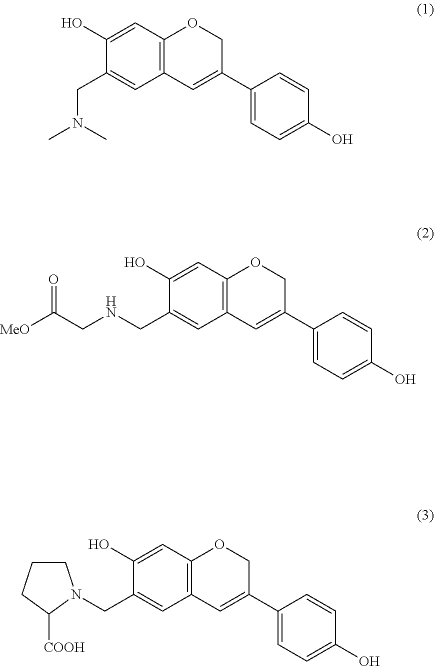

### Anti-Inflammatory Activity and Other Uses of the Compounds of Formula (I)

The inventors have discovered that compounds of the formula (I), having either an amine functionality or a nitrogen-containing ring in a side chain attached to the 6-position of the isoflavan or isoflavene nucleus, possess anti-inflammatory activity.

Accordingly, the compounds of formula (I) are useful in the prevention and/or treatment of inflammation and inflammatory diseases or disorders. Examples of inflammatory diseases and disorders include, but are not limited to: conditions associated with high estrogen levels, psoriasis and other inflammatory diseases of the skin, inflammatory lesions, fibromyalgia, sarcoidosis, systemic sclerosis, Alzheimer's disease, proliferative retinopathy, hepatitis, arthritis (including for example, osteoarthritis), inflammatory bowel disease (including, for example, forms of colitis such as ulcerative colitis and Crohn's disease), diverticulitis, ulcerative proctitis, autoimmune disorders (including, for example, systemic lupus erythematosis, rheumatoid arthritis, glomerulonephritis and Sjögren's syndrome), asthma, diseases and disorders involving pulmonary inflammation and atherosclerosis. The compounds of formula (I) may also be useful for the prevention and/or treatment of pain, oedema and/or erythema that is associated with inflammation.

Compounds of formula (I) are advantageous in the prevention and/or treatment of inflammation, inflammatory diseases and disorders in that at physiologically relevant concentrations, they are not associated with adverse cardiovascular events, as is the case with a number of other anti-inflammatory drugs currently in use. In fact, the compounds of formula (I) demonstrate cardioprotective effects and hence may be suitable for the prevention and/or treatment of cardiovascular diseases, including but not limited to: myocardial infarction, atherosclerosis, cerebrovascular disease, hypertension, angina pectoris, ischemia, coronary artery disease, congestive heart failure and blood vessel diseases.

When used for the prevention and/or treatment of inflammation and inflammatory diseases and disorders, the compounds of formula (I), and pharmaceutical compositions comprising same may be used in combination with, or include one or more other therapeutic agents, for example other anti-inflammatory agents, anticholinergic agents (particularly M1, M2, M1/M2 or M3 receptor antagonists), β2-adrenoreceptor agonists, antiinfective agents (e.g. antibiotics, antivirals), or antihistamines. Combinations may comprise a compound or compounds of formula (I) or pharmaceutically acceptable salts, solvates or physiologically functional derivatives thereof, together with a corticosteroid and/or an anticholinergic and/or a PDE-4 inhibitor.

Suitable anti-inflammatory agents include corticosteroids and NSAIDs. Suitable corticosteroids, which may be used in combination with the compounds of the formula (I) are those oral and inhaled corticosteroids and their pro-drugs which have anti-inflammatory activity. Examples include methyl prednisolone, prednisolone, dexamethasone, fluticasone propionate, 6α,9α,-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17α-carbothioic acid S-(2-oxo-tetrahydro-furan-3S-yl)ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide and butixocort propionate. Preferred corticosteroids include fluticasone propionate, and 6α,9α-difluoro-17α-[(2-furanylcarbonyl)oxy]-11β-hydroxy-16α-methyl-3-oxo-androsta-1,4-diene-17β-carbothioic acid S-fluoromethyl ester, more preferably 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Suitable NSAIDs include sodium cromoglycate, nedocromil sodium, phosphodiesterase (PDE) inhibitors (e.g. theophylline, PDE4 inhibitors or mixed PDE3/PDE4 inhibitors), leukotriene antagonists, inhibitors of leukotriene synthesis, iNOS inhibitors, tryptase and elastase inhibitors, b-2 integrin antogonists and adenosine receptor agonists or antagonists (e.g. adenosine 2α agonists), cytokine antagonists (e.g. chemokine antagonists) or inhibitors of cytokine synthesis.

The co-administration of compounds may be simultaneous or sequential. Simultaneous administration may be effected by the compounds being in the same unit dose, or in individual and discrete unit doses administered at the same or similar time. Sequential administration may be in any order as required, and may require an ongoing physiological effect of the first or initial compound to be current when the second or later compound is administered, especially where a cumulative or synergistic effect is desired.

The present inventors have also discovered that compounds of formula (I) possess potent oxidation-inhibiting properties. Accordingly, the compounds of formula (I) may be useful in a wide range of applications as antioxidants, and may conveniently be included in food stuffs or drinks and consumed accordingly.

The inventors have also found that the compounds of formula (I) may be useful in the modulation of the immune system. For example the compounds of formula (I) may be immunosuppressive and thus find utility in the treatment of conditions associated with inappropriate immune responses, for example inflammatory bowel disease and rheumatoid arthritis.

The inventors have further discovered that the compounds of formula (I) may be useful in inhibiting the proliferation of cells, and hence beneficial in the prevention and/or treatment of diseases and disorders associated with aberrant cell proliferation, for example cancer. Examples of cancers that may be treated or prevented include, but are not limited to: gastrointestinal tumours, cancer of the liver and biliary tract, pancreatic cancer, prostate cancer, testicular cancer, lung cancer, skin cancer (for example melanoma), breast cancer, non-melanoma skin cancer (for example basal cell carcinoma and squamous cell carcinoma), ovarian cancer, uterine cancer, cervical cancer, cancer of the head and neck, bladder cancer, sarcomas and osteosarcomas, Kaposi sarcoma, AIDS-related Kaposi sarcoma, renal carcinoma, leukaemia, colorectal cancer and glioma. The cancer may be a primary or secondary cancer.

In the treatment or prevention of cancer, therapeutic advantages may be obtained through combination treatment regimens. As such, methods of treatment of cancer according to the present invention may be used in conjunction with other therapies, such as radiotherapy, chemotherapy, surgery, or other forms of medical intervention. Non-limiting examples of suitable chemotherapeutic and other anti-cancer agents include: taxol, fluorouracil, cisplatin, oxaliplatin, α-interferon, vincristine, vinblastine, angioinhibins, doxorubicin, bleomycin, mitomycin C, phenoxodiol, NV-128, methramycin, TNP-470, pentosan polysulfate, tamoxifen, LM-609, CM-101 and SU-101.

The co-administration of compounds of the formula (I) and chemotherapeutic or other anti-cancer agents may be simultaneous or sequential. Simultaneous administration may be effected by a compound of the formula (I) being in the same unit dose as a chemotherapeutic or other anti-cancer agent, or the compound of the formula (I) and the chemotherapeutic or other anti-cancer agents may be present in individual and discrete unit doses administered at the same, or at a similar time. Sequential administration may be in any order as required, and may require an ongoing physiological effect of the first or initial compound to be current when the second or later compound is administered, especially where a cumulative or synergistic effect is desired.

### Pharmaceutical Compositions and Routes of Administration

The compounds of formula (I) are useful as therapeutic agents in the treatment or prevention of various diseases or conditions in a subject. The compounds of formula (I) may be administered to a subject in the form of pharmaceutical compositions.

Pharmaceutical compositions include those suitable for oral, parenteral (including subcutaneous, intradermal, intramuscular, intravenous and intraarticular), inhalation (including use of metered dose pressurised aerosols, nebulisers or insufflators), rectal and topical (including dermal, buccal, sublingual and intraocular) administration. The most suitable route may depend upon, for example, the condition and disorder of the recipient.

The compositions may conveniently be presented in unit dosage form and may be prepared by any of the methods well known in the art of pharmacy. All methods include the step of bringing one or more compounds of the formula (I) into association with a carrier which constitutes one or more accessory ingredients. In general, the compositions are prepared by uniformly and intimately bringing into association one or more compounds of the formula (I) with a liquid carrier or finely divided solid carrier, or both and then, if necessary, shaping the product into the desired composition.

Generally, an effective dosage of a compound of the formula (I) is expected to be in the range of about 0.0001 mg to about 1000 mg per kg body weight per 24 hours; about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours, or about 1.0 mg to about 250 mg per kg body weight per 24 hours. More typically, an effective dose range is expected to be in the range of about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours, or about 5.0 mg to about 15 mg per kg body weight per 24 hours.

Alternatively, an effective dosage may be up to about 500 mg/m². Generally, an effective dosage is expected to be in the range of about 25 to about 500 mg/m², about 25 to about 350 mg/m², about 25 to about 300 mg/m², about 25 to about 250 mg/m², about 50 to about 250 mg/m², or about 75 to about 150 mg/m².

Compositions suitable for buccal (sublingual) administration include lozenges comprising a compound of the formula (I) in a flavoured base, usually sucrose and acacia or tragacanth; and pastilles comprising a compound of the formula (I) in an inert base such as gelatine and glycerin or sucrose and acacia.

Compositions suitable for oral administration may be presented as discrete units such as gelatine or HPMC capsules, cachets or tablets, each containing a predetermined amount of a compound of formula (I) as a powder, granules, as a solution or a suspension in an aqueous liquid or a non-aqueous liquid, or as an oil-in-water liquid emulsion or a water-in-oil liquid emulsion. The compound of formula (I) may also be present as a paste.

When compounds of the formula (I) are formulated as capsules, the compound may be formulated with one or more pharmaceutically acceptable carriers such as starch, lactose, microcrystalline cellulose, silicon dioxide and/or a cyclic oligosaccaride such as cyclodextrin. Additional ingredients may include lubricants such as magnesium stearate and/or calcium stearate. Suitable cyclodextrins include α-cyclodextrin, β-cyclodextrin, γ-cyclodextrin, 2-hydroxyethyl-β-cyclodextrin, 2-hydroxypropyl-cyclodextrin, 3-hydroxypropyl-β-cyclodextrin and tri-methyl-β-cyclodextrin. The cyclodextrin may be hydroxypropyl-β-cyclodextrin. Suitable derivatives of cyclodextrins include Captisol® a sulfobutyl ether derivative of cyclodextrin and analogues thereof as described in U.S. Pat. No. 5,134,127.

Tablets may be prepared by compression or moulding, optionally with one or more accessory ingredients. Compressed tablets may be prepared by compressing in a suitable machine the compound of formula (I) in a free-flowing form such as a powder or granules, optionally mixed with a binder, lubricant (for example magnesium stearate or calcium stearate), inert diluent or a surface active/dispersing agent. Moulded tablets may be made by moulding a mixture of the powdered compound of formula (I) moistened with an inert liquid diluent, in a suitable machine. The tablets may optionally be coated, for example, with an enteric coating and may be formulated so as to provide slow or controlled release of the compound of formula (I) therein.

Compositions for parenteral administration include aqueous and non-aqueous sterile injectable solutions which may contain anti-oxidants, buffers, bacteriostats and solutes which render the formulation isotonic with the blood of the intended recipient, and which may include suspending agents and thickening agents. A parenteral composition may comprise a cyclic oligosaccaride such as hydroxypropyl-β-cyclodextrin. The compositions may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described.

Dry powder compositions for topical delivery to the lung by inhalation may, for example, be presented in capsules and cartridges of, for example gelatine, or blisters or for example laminated aluminium foil, for use in an inhaler or insufflator. Compositions generally contain a powder mix for inhalation of the one or more compounds of the formula (I) and a suitable powder base (carrier substance) such as lactose or starch. Use of lactose is preferred. Each capsule or cartridge may generally contain between 20 μg-10 mg of a compound of formula (I), optionally in combination with another therapeutically active ingredient. Alternatively, the compound or compounds of the formula (I) may be presented without excipients. Packaging of the composition may be for unit dose or multi-dose delivery.

Compositions suitable for transdermal administration may be presented as discrete patches adapted to remain in intimate contact with the epidermis of the recipient for a prolonged period of time. Such patches suitably comprise the compound of the formula (I) as an optionally buffered aqueous solution of, for example, 0.1M to 0.2 M concentration with respect to the compound.

Compositions suitable for transdermal administration may also be delivered by iontophoresis, and typically take the form of an optionally buffered aqueous solution of the active compound. Suitable compositions comprise citrate or Bis/Tris buffer (pH 6) or ethanol/water and contain from 0.1M to 0.2 M of a compound of the formula (I).

Spray compositions for topical delivery to the lung by inhalation may, for example be formulated as aqueous solutions or suspensions or as aerosols, suspensions or solutions delivered from pressurised packs, such as a metered dose inhaler, with the use of a suitable liquefied propellant. Suitable propellants include a fluorocarbon or a hydrogen-containing chlorofluorocarbon or mixtures thereof, particularly hydrofluoroalkanes, e.g. dichlorodifluoromethane, trichlorofluoromethane, dichlorotetrafluoroethane, especially 1,1,1,2-tetrafluoroethane, 1,1,2,2,3,3,3-heptafluoro-n-propane or a mixture thereof. Carbon dioxide or other suitable gas may also be used as propellant. The aerosol composition may be excipient free or may optionally contain additional composition excipients well known in the art, such as surfactants e.g. oleic acid or lecithin and cosolvents e.g. ethanol. Pressurised compositions will generally be retained in a canister (e.g. an aluminium canister) closed with a valve (e.g. a metering valve) and fitted into an actuator provided with a mouthpiece.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. When the excipient is lactose it will typically be present as milled lactose, wherein not more than 85% of lactose particles will have a MMD of 60-90 μm and not less than 15% will have a MMD of less than 15 μm.

Compositions for rectal administration may be presented as a suppository with carriers such as cocoa butter or polyethylene glycol, or as an enema wherein the carrier is an isotonic liquid such as saline. Additional components of the compositions may include a cyclic oligosaccaride, for example, a cyclodextrin, as described above, such as hydroxypropyl-β-cyclodextrin, one or more surfactants, buffer salts or acid or alkali to adjust the pH, isotonicity adjusting agents and/or anti-oxidants.

Compositions suitable for topical administration to the skin preferably take the form of an ointment, cream, lotion, paste, gel, spray, aerosol, or oil. Carriers which may be used include Vasoline, lanoline, polyethylene glycols, alcohols, and combination of two or more thereof. The compound of the formula (I) is generally present at a concentration of from 0.1% to 5% w/w, or from 0.5% to 2% w/w. Examples of such compositions include cosmetic skin creams.

The compounds of the formula (I) may be provided in the form of food stuffs, such as being added to, admixed into, coated, combined or otherwise added to a food stuff. The term “food stuff” is used in its widest possible sense and includes liquid compositions such as drinks, including dairy products and other foods, such as health bars, desserts, etc. Food compositions comprising compounds of the formula (I) can be readily prepared according to standard practices.

The production of pharmaceutical compositions for the treatment of the therapeutic indications herein described are typically prepared by admixture of the compounds of the formula (I) with one or more pharmaceutically or veterinary acceptable carriers and/or excipients as are well known in the art.

The carrier must, of course, be acceptable in the sense of being compatible with any other ingredients in the composition and must not be deleterious to the subject. The carrier or excipient may be a solid or a liquid, or both, and is preferably formulated with the compound as a unit-dose, for example, a tablet, which may contain up to 100% by weight of the active compound, preferably from 0.5% to 75% by weight of the compound of the formula (I).

The composition may also be administered or delivered to target cells in the form of liposomes. Liposomes are generally derived from phospholipids or other lipid substances, and are formed by mono- or multi-lamellar hydrated liquid crystals that are dispersed in an aqueous medium. Specific examples of liposomes used in administering or delivering a composition to target cells are synthetic cholesterol (Sigma), 1,2-distearoyl-sn-glycero-3-phosphocholine (DSPC; Avanti Polar Lipids), 3-N-[(-methoxy poly(ethylene glycol)2000)carbamoyl]-1,2-dimyrestyloxy-propylamine (PEG-cDMA), or 1,2-di-o-octadecenyl-3-(N,N-dimethyl)aminopropane (DODMA).

The compositions may also be administered in the form of microparticles. Biodegradable microparticles formed from polylactide (PLA), polylactide-co-glycolide (PLGA), and \acute{ε}-caprolactone have been extensively used as drug carriers to increase plasma half life and thereby prolong efficacy (R. Kumar, M., 2000, J. Pharm. Pharmaceut. Sci. 3(2) 234-258).

The compositions may incorporate a controlled release matrix that is composed of sucrose acetate isobutyrate (SAIB) and organic solvent or organic solvent mixtures. Polymer additives may be added to the vehicle as a release modifier to further increase the viscosity and slow down the release rate. A compound of the formula (I) may be added to the SAIB delivery vehicle to form SAIB solution or suspension compositions. When the formulation is injected subcutaneously, the solvent diffuses from the matrix allowing the SAIB-drug or SAIB-drug-polymer mixtures to set up as an in situ forming depot.

The present invention will now be described with reference to specific examples, which should not be construed as in any way limiting the scope of the invention.

### EXAMPLES

### Example 1

### Preparation of Compounds

Compound (1) was synthesised as follows. Dehydroequol (6.5 g, 27.1 mmol) was weighed into a 250 mL round bottom flask and dissolved in absolute ethanol (125 mL). The solution was cooled to 0° C. after which N,N,N′,N′-tetramethyldiaminomethane (4.7 mL, 34.9 mmol) was added, followed by formaldehyde (18 mL, 37% aq. solution). The mixture was stirred at room temperature overnight after which a white precipitate had formed that was collected by suction filtration and dried under high vacuum to yield compound (1). Yield 5.53 g, 69%.

¹H NMR (400 MHz, d6-DMSO) δ 2.22 (s, 6H, 2×CH₃), 3.48 (s, 2H, —CH₂—), 5.01 (d, 2H, J=1.04 Hz, H2), 6.21 (s, 1H, H4), 6.73 (s, 1H, H8), 6.77 (d, 2H, J=8.7 Hz, H3′, H5′), 6.83 (s, 1H, H5), 7.32 (d, 2H, J=8.7 Hz, H2′, H6′).

Compound (2) was synthesised as follows. Dehydroequol (0.24 g, 1.0 mmol) was dissolved in ethanol (ca. 10 mL) and stirred in an ice bath. The flask was sealed with a septum to prevent escape of formaldehyde. Glycine methyl ester hydrochloride (0.25 g, 2.0 mmol), triethylamine (0.28 ml, 2.0 mmol) and 37% formaldehyde solution (0.35 mL, 4.0 mmol) were added to the reaction mixture, and the mixture was allowed to stir for 24 hours at room temperature. Ethanol was removed under vacuum and the resulting residue chromatographed on silica to yield compound (2) (0.17 g, 51%).

¹H NMR (400 MHz, d6-DMSO) δ 3.64 (s, 3H, OCH₃), 3.92 (s, 2H, NCH₂), 4.82 (s, 2H, NCH₂), 5.02 (s, 2H, H2), 6.22 (s, 1H, H8), 6.74 (s, 1H, H4 or H5), 6.77 (s, 1H, H4 or H5), 6.78 (d, 2H, J=8.7 Hz, H3′ and H5′), 7.34 (d, 2H, J=8.7 Hz, H2′ and H6′), 9.64 (br s, 2H, OH).

Compound (3) was synthesised as follows. L-Proline (0.46 g, 4.00 mmol) and 37% formaldehyde (0.31 mL, 4.16 mmol) in water (ca. 20 mL) was added to a stirred solution of dehydroequol (0.50 g, 2.08 mmol) in ethanol (ca. 40 mL). The mixture was then refluxed at 70-80° C. for ˜7 hours. The mixture was cooled to room temperature before the mixture was concentrated in vacuo leaving a pink solid. This solid was collected under suction and the filtrate evaporated to dryness to yield a second crop (combined yield of compound (3) 0.33 g, 92%), m.p. 240° C. (dec.).

¹H NMR (300 MHz, DMSO-d₆): δ 7.31 (d, 2H, J=10.2, Hz, H2′, H6′), 6.98 (s, 1H, H5), 6.76 (d, 2H, J=8.6 Hz, H3′, H5′), 6.72 (s, 1H, H4), 6.28 (s, 1H, H8), 5.02 (s, 2H, H2), 4.08 (d, 1H, J=12.8 Hz, Ar—CHₐ—N) 3.74 (d, 1H, J=13.2 Hz, Ar—CH_{b}—N) 2.70 (dd, 1H, J=9.4 Hz, J=17.3 Hz, —CH—COOH) 2.18-2.08 (m, 1H, —N—CH₂—CH₂) 1.95-1.66 (m, 4H, —CH₂—CH₂—CH₂—CH—COOH).

¹³C NMR (75.6 MHz, DMSO-d₆): δ 171.99, —C═O; 157.59, ArC; 157.46, ArC; 154.22, ArC—OH; 129.11, ArCH; 128.30, ArC; 127.42, ArC; 126.10, ArCH; 116.67, ArCH; 115.88, ArCH; 115.05, ArC; 113.89, ArC; 102.79, ArCH; 72.65, Ar—CH₂—O; —CH—COOH; 53.93, —N—CH₂; 53.16, Ar—CH₂—N, 28.98, —CH—CH₂—CH₂; 23.52, —CH₂—CH₂—CH₂.

IR (KBr): υₘₐₓ 3422, 3104, 1616, 1508, 1458, 1396, 1312, 1272, 1158, 1132 cm⁻¹.

UV/Vis (CH₃OH): λₘₐₓ 336 nm (ε24101 cm⁻¹M⁻¹), 253 nm (ε17194 cm⁻¹M⁻¹), 214 nm (ε26232 cm⁻¹M⁻¹), 202 nm (ε27150 cm⁻¹M⁻¹).

HRMS calculated d for C₂₁H₂₁NO₅Na⁺: 390.13119. found 390.13192.

Microanalysis: Found C, 67.79; H, 5.94; N, 3.74. calculated C, 67.68; H, 6.20; N, 3.59% for C₂₁H₂₁NO₅).

### Example 2

### Anti-Inflammatory Activity

### 2.1 Effect on NFκB Production in the Transfected Human Macrophage Cell Line THP-1

NFκB is a ubiquitous transcription factor central to cellular responses to stimuli such as stress, proinflammatory cytokines (eg IL-1 or TNF-α), free radicals, ultraviolet irradiation, and bacterial or viral antigens. Its inhibition provides an anti-inflammatory strategy.

### Methods

The assay utilizes a genetically modified THP-1 cell line and GeneBLAzer® beta-lactamase technology (Invitrogen Corp). The human THP-1 monocyte/macrophages contain a stably-transfected beta-lactamase reporter gene under control of the NFkB response element. They respond to stimulation with TNFα, which leads to activation of the NFkB signaling pathway. Co-incubation of cells with TNFα and test material allows quantitative determination of the ability of test material to inhibit TNFα-stimulated beta-lactamase production. An inflammatory index was calculated as the ratio of beta-lactamase product to beta-lactamase substrate.

In brief, genetically modified THP-1 cells were seeded into wells of a 96-well plate (50×10³ cells/well) in the presence of RPMI 1640 medium (70 μl). TNFα was added to each well (10 μl) to give a final concentration of 7.5 ng/ml. Dialyzed bovine serum was added (10 μl). Test compounds were dissolved in DMSO (10 μL) (5 wells). Each plate contained a no-cell control (4 wells), a no-serum control (4 wells) and two serum controls. Plates were incubated for 5 h at 37° C. to allow for NFkB-stimulated beta-lactamase production. LiveBLAzer™ FRET B/G Substrate (CCF4-AM) substrate was then added to the assay. CCF4-AM is a Förster resonance energy transfer (FRET) based substrate for beta-lactamase developed by Invitrogen Corp. Once CCFA-AM enters a cell, it is converted to negatively charged CCF4 by endogenous esterases. Excitation of this substrate at 409 nm leads to efficient FRET between the coumarin and fluorescein moieties, resulting in a green fluorescence detectable at 530 nm. The presence of beta-lactamase leads to cleavage of CCF4 and results in a loss of FRET, resulting in a robust blue fluorescent signal detectable at 460 nm. Thus, activity of beta-lactamase (a marker of NFkB-promoter activity) is measured as a product to substrate ratio (blue/green fluorescence ratio: 460 nm/530 nm). The determination of inflammatory index had a within-plate CV of 2.1% and a between-plate CV of 8.9%.

### Results

As shown in Table 1 below, compound (1) significantly reduced the promoter activity of NFκB at 10 μM and 100 μM. It did so in the absence of cytotoxicity. Compound (2) was active at the highest concentration only, and again in the absence of cytotoxicity.

TABLE 1
Effect of test compounds on NFκB promoter activity in THP-1 cells
Inflammatory index

| 0 μM | 10 μM | 30 μM | 100 μM |
|---|---|---|---|
| Vehicle | 8.92 ± 0.22 | 11.56 ± 0.49 | 9.48 ± 0.14 |
| Compound (1) | 9.12 ± 0.26 | 10.05 ± 0.36 *** | 2.75 ± 0.11 *** |
| Compound (3) | 8.88 ± 0.05 | 11.45 ± 0.86 | 8.59 ± 0.24 *** |

Significantly different from control cells incubated without compound:
* P < 0.05;
** P < 0.01;
*** P < 0.001.

These results suggest that both compounds (1) and (3) possess activity integral to modulating inflammation.

### 2.2 Effect on Adhesion Molecule Expression in Arterial Cells

Inflammation involves the recruitment of inflammatory cells from the circulation and their transendothelial migration. This process is predominantly mediated by cellular adhesion molecules, which are expressed on the vascular endothelium and on circulating leukocytes in response to several inflammatory stimuli. Vascular cell adhesion molecule-1 (VCAM-1) induces firm adhesion of inflammatory cells at the vascular surface. Consequently, inhibition of VCAM-1 is a potential therapeutic target for the control of inflammation in general and arthritis in particular.

### Methods

Inhibition of TNFα-stimulated endothelial cell activation was assessed by measuring surface expression of cell adhesion molecules with an ELISA method. Human arterial endothelial cells (HAEC) in growth medium (Cell Applications Inc.) were seeded into 96-well plates at a density of 10,000 cells per well. Plates were incubated overnight at 37° C. in a humidified incubator to allow for cells to become confluent. On the morning of the experiment, TNFα (10 μl, 2 ng/ml) was added to each well, which contained 100 μl of medium. Test compounds were diluted in DMSO-containing medium (2.5% DMSO) to give a concentration of 100 and 300 μM. They were added to wells so that final concentrations were 10 and 30 μM. DMSO-containing medium alone was added to zero concentration control wells. All samples were measured in quadruplicate (4 wells per treatment).

After incubation with a compound of the formula (I), the medium was removed and cells were probed with either non-specific IgG or specific mouse antibodies (VCAM (BD Biosciences −0.1 μg in 100 μL buffered saline with 10% heat-inactivated human serum)).

Adhesion molecule expression was detected by addition of sheep anti-mouse antibody/horseradish peroxidase conjugate. Plates were allowed to stand for 30 minutes—monolayers were then washed, and sheep anti-mouse antibody/horseradish peroxidase conjugate (1:500 in 100 μL HBSS with 10% heat-inactivated human serum and 0.05% Tween 20) was added and left for 30 minutes. After further washing, 150 μL, ABTS substrate (Kirkegaard and Perry Laboratories) was added to each well and allowed to develop for 15 minutes. Optical density was measured at 405 nm with an ELISA reader (Titertek Multiscan, Flow Laboratories).

### Results

As shown below in Tables 2 and 3, compounds (1) and (3) significantly inhibited TNFα-induced VCAM expression at both concentrations.

TABLE 2
Effect of compound (1) on VCAM-1 expression in HAECs
Absorbance

| 0 μM | 10 μM | 30 μM |
|---|---|---|
| Vehicle | 0.214 ± 0.023 | 0.228 ± 0.011 |
| Compound (1) | 0.164 ± 0.012 ** | 0.208 ± 0.008 * |

TABLE 3
Effect of compound (3) on VCAM-1 expression in HAECs
Absorbance

| 0 μM | 10 μM | 30 μM |
|---|---|---|
| Vehicle | 0.204 ± 0.016 | 0.272 ± 0.033 |
| Compound (3) | 0.161 ± 0.16 ** | 0.215 ± 0.018 * |

Significantly different from control cells incubated without compound:
* P < 0.05;
** P < 0.01;
*** P < 0.001.

These results suggest that both compounds (1) and (3) may reduce the recruitment and migration of leucocytes involved in the inflammatory response.

### 2.3 Effect on Lipoxygenase

Leukotrienes (LTs) are eicosanoids, a family of molecules derived from arachidonic acid (AA). Unlike the PGs and the TXs, which are products of the COX pathway, LTs are products of the 5-lipoxygenase (5-LO) pathway. LTs play a role in allergic and inflammatory diseases, amplifying inflammation by causing increased vascular permeability, vasodilation and smooth muscle contraction. In addition, they are potent chemotactic agents. Moreover, inhibition of 5-LO indirectly reduces the expression of TNFα. Inhibition of LTs is an anti-inflammatory strategy.

### Methods

The pathway for LTB₄ synthesis involves initial release of AA from phospholipids by a Ca-dependent PLA₂. The free AA is then oxygenated at by 5-LO (requiring enzyme activation by FLAP) to generate an epoxide intermediate (LTA₄). LTA₄ is then converted to LTB₄ by LTA₄ hydrolase. LTB₄ is metabolised (and deactivated) by a cytochrome P-(CYP) 450 ω-hydrolase to produce 20-hydroxy and 20-carboxy metabolites. These metabolites are also measured in the HPLC assay.

Neutrophils were isolated from citrated human venous blood to >90% purity by centrifugation through Ficoll, dextran sedimentation and lysis of erythrocytes (Boyum 1986). Cells were washed in HEPES buffered Hanks solution (HBHS) and then suspended at 4.5 million cells/mL in HBHS containing 0.1% BSA (HBHS+BSA).

Experiments had been carried out previously to optimise the stimulation of neutrophils by calcium ionophore. At 37° C., 900 μL of cell suspension (4 million cells) was incubated with 3′,7-dihydroxyisoflav-3-ene (or vehicle) in 10 μL DMSO for 5 min before addition of 100 μL of 25 ng/μL calcium ionophore (free acid form, Sigma) with 0.5% DMSO in HBHS containing 0.1% bovine serum albumin (HBHS+BSA). The cells were incubated for 10 min then pelleted by centrifugation at 1200× g at 4° C. for 5 min, and the cell free supernatant used to quantitate the levels of LTB₄ and its metabolites.

To each 900 μL aliquot of the supernatant, 25 μL of 2.5 ng/μL prostaglandin B₂ (PGB₂) in ethanol was added as internal standard. The solution was acidified to pH with 2M formic acid and the mixture extracted with 2 mL ethyl acetate and vigorous vortexing. The organic layer was collected and dried under nitrogen in a glass vial before being reconstituted in 50 μL of the reconstitution solution (water:methanol:acetonitrile at 2:1:1).

Analysis was carried out using a HPLC system with a 125-4 LiChrospher®100 RP-18 (5 μm) column (Agilent Technologies) and a gradient system adapted from a published method (Mita et al. 1988) to separate LTB₄, its oxidation products 20-hydroxy LTB₄ (20-OH-LTB₄) and 20-carboxy LTB₄ (20-COOH-LTB₄), as well as PGB₂. At 1 ml/min flow rate, a combination of three different mobile phase solutions were used. UV absorbance was monitored at 270 nm, and LTB₄ and its metabolites were quantitated by comparison of peak areas with that of internal standard and a standard curve prepared earlier.

### Results

As shown in Table 4 below, compound (1) was active in inhibiting the synthesis of LTB₄ and its metabolites. The IC₅₀ for the production of LTB₄ was 4.3 μM. As also shown in Table 4, compound (3) was active in inhibiting the synthesis of LTB₄ and the IC₅₀ for its production was 5.4 μM. Whilst it inhibited the production of 20-OH-LTB₄, the production of 20-COOH-LTB₄ was enhanced.

The maximum release of LTB₄, 20-OH-LTB₄ and 20-COOH-LTB₄ produced by compounds (1) and (3) was compared to that of vehicle control.

TABLE 4
Effect of compounds (1) and (3) on the
synthesis of LTB₄ and its metabolites

| | | % of maximum release | | |
|---|---|---|---|---|
| Compound | μM | LTB₄ | 20-COOH-LTB₄ | 20-OH-LTB₄ |
| (1) | 10 | 0.0 | 0.0 | 0.0 |
| | 1 | 78.1 | 99.6 | 77.7 |
| | 0.1 | 94.3 | 82.0 | 95.2 |
| (3) | 10 | 0.0 | 145.2 | 6.1 |
| | 1 | 97.0 | 105.7 | 99.0 |
| | 0.1 | 93.6 | 114.9 | 82.8 |

Overall, the cell viability was around 75%-85%, using an aliquot of the reaction mixture and incubation cells with the test compounds for 5 minutes. Cell viability of neutrophils incubated with test compounds was similar to that of controls.

These results indicate that both compounds (1) and (3) possess lipoxygenase inhibitory activity.

### 2.4 Effect on the Production of Nitric Oxide

Nitric oxide (NO), a molecular messenger synthesized by nitric oxide synthase (NOS) from L-arginine and molecular oxygen, is involved in a number of physiological and pathological processes. Three structurally distinct isoforms of NOS have been identified: neuronal (nNOS), endothelial (eNOS) and inducible (iNOS). Excess NO produced by iNOS has been implicated in inflammation. For example, in arthritic joints, NO causes apoptosis and dedifferentiation of articular chondrocytes by the modulation of extracellular signal-regulated kinase (ERK), p38 kinase, and protein kinase C (PKC). In contrast, NO produced by eNOS has a physiological role in maintaining vascular tone. eNOS-derived NO also regulates endothelial cell adhesion molecule expression, leukocyte adhesion, and extravasation-significant increases in constitutive expression of adhesion molecules ICAM-1 and P-selectin, leukocyte rolling, adhesion, and extravasation were seen in the vasculature of tissues from eNOS knock-out mice compared to their wild-type controls. Accordingly, selective inhibition of iNOS and upregulation of eNOS would be an advantageous as an anti-inflammatory strategy, as well as provide a cardioprotective effect.

### Methods

The mouse macrophage cell line RAW 264.7 was cultured in DMEM supplemented with foetal calf serum (FCS), 2 mM glutamine and 50 U/ml penicillin/streptomycin. Cells were treated with either the compounds of the formula (I) (in 0.025% DMSO) or vehicle alone, and added one hour before 50 ng/ml LPS, which induces iNOS and the production of NO. After incubation for 16 hrs, culture media was collected. Nitrite concentration is a quantitative indicator of NO production and was determined by the Griess Reaction. Briefly, 100 μL of Griess reagent was added to 50 μL of each supernatant in duplicate. The absorbance at 550 nm was measured (Molecular Devices, SpectraMax 250 microplate spectrophotometer, CA, USA), and nitrite concentrations were determined against a standard curve of sodium nitrite.

### Results

As can be seen from Table 5 below, compounds (1), (2) and (3) had some inhibitory effect on NO synthesis in a dose responsive manner. In the case of compound (1), this effect may have been influenced by toxicity in RAW 264.7, where the IC₅₀ is 53.9±1.2 μM.

TABLE 5
Effect of test compounds on NO synthesis in RAW 264.7

| | NO (μM) | | |
|---|---|---|---|
| Compound | 0 μM | 1 μM | 10 μM |
| (1) | 17.38 ± 0.44 | 17.27 ± 0 | 5.29 ± 0.15 |
| (2) | 17.17 ± 0.15 | 16.41 ± 0.0 | 10.22 ± 0.15 |
| (3) | 35.51 ± 0.16 | 31.33 ± 0.16 | 21.79 ± 0.82 |

2.5 Effect on Expression of Endothelial Nitric Oxide Synthase (eNOS)

### Methods

HCAECs were grown as described above. Because cell viability was less than 100% at 30 and 100 μM, eNOS experiments were conducted at one concentration (10 μM). After incubation, total RNA was extracted using TRI reagent (Sigma, St Louis, Mo., USA), following the manufacturer's protocol. RNA was quantitated and normalized to 100 ng/μl using the SYBR Green II assay (Molecular Probes, Eugene, Oreg., USA) before being reverse transcribed using iScript (Bio-Rad, Hercules, Calif., USA). eNOS (sense 5′-CCA TCT ACA GCT TTC CGG CGC-3′ and antisense 5′-CTC TGG GGT GGC CTT CAG CA-3′) and 18S (sense 5′-CGG CTA CCA CAT CCA AGG AA-3′ and antisense 5′-GCT GGA ATT ACC GCG GCT-3′) mRNA levels were determined by real-time PCR using iQ SYBR Green Supermix (Bio-Rad) in an iCycler iQ RealTime thermocyler detection system (Bio-Rad Laboratories). The cycling parameters were 95° C. for 30 seconds, 62° C. for 30 seconds, and 72° C. for 30 seconds for 40 cycles, and real-time data was collected at each cycle. There were six replicates.

### Results

Compounds (1) and (3) were examined at a single concentration of 10 μM. At 10 μM and for the 24 hr incubation period, cell viability was unaffected. As seen below in Table 6, both compounds significantly increased the expression of eNOS-compound (1) by an average of 45% and compound (3) by 325%.

TABLE 6
Relative expression of eNOS mRNA

| | Increase in expression relative to control cells (p value) | | |
|---|---|---|---|
| Compound | EXPT 1 | EXPT 2 | EXPT 3 |
| (1) | 52 ± 25% (0.0001) | 53 ± 49 (0.009) | 30 ± 30 (0.014) |
| (3) | 372 ± 138% (0.00001) | 277 ± 212 (0.002) | — |

### 2.6 Activity in the Rat Adjuvant-Induced Arthritis Model

Adjuvant-induced arthritis in genetically susceptible rodents is a well accepted animal model of chronic joint inflammation such as that experienced in rheumatoid arthritis. It is responsive to anti-inflammatory and immunosuppressive agents.

### Methods

Male Dark Agouti (DA) strain (DA.CD45.1) rats were fed with either compound (1)-treated feed or placebo-treated feed for seven days prior to the injection of Complete Freund's adjuvant (0.1 ml) into the base of the tail. The treated feeds were continued throughout the experiment. Arthritis, which became evident at Day 8 was subjectively scored each day by an operator blinded to the identity of treatments and using a scoring system as follows:
0 (no evidence of arthritis);
1 (1 or 2 red, swollen joints but no other swelling);
2 (carpus or tarsus swollen or more than 2 small joints swollen);
3 (some red swollen joints and carpus or tarsus swollen, but not global swelling);
4 (severe global swelling of the entire paw).

Therefore, the disease score for individual rats ranged between 0 and 16. Rats (n=8 per group) were killed on Day 12. Data were analysed using a two way ANOVA (Prism 4 for Windows, GraphPad Software Inc).

### Results

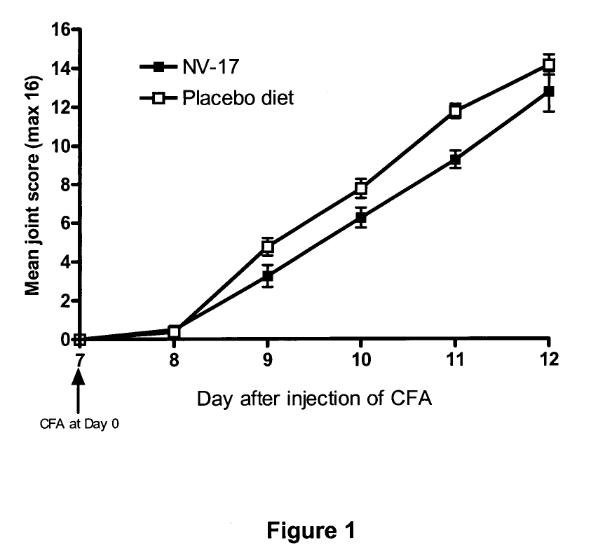

As shown below in Table 7, treatment with compound (1) caused a statistically significant (p=0.008) reduction in arthritis score when compared with treatment with placebo feed (see FIG. 1).

TABLE 7
Joint score day by day (mean ± SD)

| Day of expt | Compound (1) | Placebo |
|---|---|---|
| 7 | 0 | 0 |
| 8 | 0.5 ± 0.53 | 0.38 ± 0.52 |
| 9 | 3.25 ± 1.58 | 4.75 ± 1.28 |
| 10 | 6.25 ± 1.49 | 7.75 ± 1.39 |
| 11 | 9.25 ± 1.28 | 11.75 ± 1.04 |
| 12 | 12.75 ± 2.92 | 14.13 ± 1.46 |

### 2.7 Anti-Inflammatory Activity in the Rat Air Pouch Assay

An alternative assay used to measure anti-inflammatory efficacy is the air pouch model which involves the repeated subcutaneous injection of air into the dorsum of rats followed 24 h later by the intrapouch injection of an inflammatory stimulus (Gilroy et al. 1998).

### Methods

Air pouches were raised on the dorsum of female Dark Agouti rats, approximately seven weeks of age. To promote the formation of a cellular membrane lining the inside of each pouch, the pouches were maintained by re-inflating on days 2 and 5 after the initial injection of air. On re-inflation, the pouch was first deflated to ensure the needle was positioned correctly, before being re-inflated with 2 mL of sterile air. Using this protocol, the pouches remained inflated until use on day 7, when they were injected with 0.5 ml of either test compound or vehicle control. After 15 min, air pouches were injected with serum-treated zymozan (STZ—500 μg). Lavage of the air pouch (4×2 ml lavages) was performed at 4 h and leucocytes counted, after which the rats were killed, the air pouch excised and processed in formalin for histology. The sections were blinded to the person counting. Using a graticule with 100 squares and the 40× objective, the number of polymorphs (PMN) was counted in the pouch lining at 10 different and non-adjacent sites. Group sizes were 5-6 rats. Data were analysed for statistical significance within each experiment and using an unpaired t test. Compounds (1) and (3) were examined in this assay.

### Results

In pouches treated with compound (1), both the number of exudate cells in the pouch cavity and the number of extravasated PMN in the pouch wall were more than 3-fold less in the treated rats compared with the controls (see Table 8 below). The differences were highly significant statistically (P<0.01), demonstrating an anti-inflammatory effect.

In the case of compound (3), the mean number of exudate cells was similar for both the treatment and control group (see Table 8 below). There was a modest decrease in the number of PMN in the pouch wall of the treated group, but this was not significant statistically.

TABLE 8
Effect of test compounds on the number of cells in the exudate of
the air-pouch cavity and on the number of PMN in the air pouch wall

| | Cells in lavage fluid | PMN in tissue sections |
|---|---|---|
| | (×10⁻⁷) | (per 100 squares) |
| Compound 1 | 0.43 ± 0.21^{b} | 13.03 ± 7.10 |
| Control^{a} | 1.48 ± 0.61 | 41.53 ± 15.28 |
| | P = 0.0023^{c} | P = 0.0018 |
| Compound 3 | 1.73 ± 0.84 | 35.85 ± 12.89 |
| Control | 1.69 ± 0.29 | 45.75 ± 4.63 |
| | P = 0.9004 | P = 0.1139 (two-tailed) |
| | | P = 0.0570 (one-tailed) |

^{a}Control was DMSO/PBS, the vehicle for the test compounds. The ratio of DMSO/PBS was 1:100.
^{b}mean ± SD.
^{c}unpaired t-test (2-tailed).

### 2.8 Anti-Inflammatory Activity in Murine Ear Inflammation Assay

Compounds (1) and (3) were examined for their ability to inhibit ear swelling in mice induced by the topical application of several inflammogens-arachidonic acid (AA) and 4-β-phorbol 12-myristate 13-acetate (PMA).

The inflammatory response due AA, the immediate precursor of the eicosanoids, is due to formation of AA metabolites via both the COX and LO pathways. AA induces an early (10-15 min) increase in both PGE₂ and LTC₄ synthesis which precedes the increase in ear thickness.

Inflammation induced by PMA involves activation of protein kinase C (PKC), a phospholipid-dependent protein enzyme which plays a key role in a range of signal induction processes. In other words, PMA is a PKC activator. PKC mediates activation of phospholipase A2, resulting in the release of free AA and the subsequent synthesis of leukotrienes (LTs) and prostaglandins (PGs). The inflammation is primarily mediated by PGE₂, as levels of PGE₂ but not LTB₄ and LTC₄ are elevated in the ears of PMA-treated mice.

### Methods

Groups of 5-6 female BALB/c mice (ARC, WA, Australia), weighing 15-21 g were injected intraperitoneally (i/p) with selected compounds of the formula (I) at 25 mg/kg delivered in polyethylene glycol (PEG) 400: phosphate buffered saline (PBS) 1:1 or ethanol: propanediol:PBS 4:9:7 either 30 min prior to or immediately before the inflammogen was applied to the ears. Mice were anaesthetised using isoflurane and baseline thickness of both ears was measured using a spring micrometer. Each mouse received a total of 204 of either AA in ethanol (50 mg/ml or 200 mg/ml) or PMA in either ethanol or acetone (0.2 mg/ml) applied to the inner and outer surfaces of each pinna (i.e. 0.5 mg or 2 mg AA or 2 μg PMA per ear). Mice were anaesthetised again to re-measure the ears at 1 hr post-AA application and 5 hr after PMA.

The difference in ear swelling pre- and post-application of inflammogen for each ear was calculated, and the average for the two ears of each mouse taken. The difference in mean swelling of each test group compared to the group given vehicle alone was calculated using a general ANOVA using Dunnett's Multiple Comparison test when multiple compounds were tested in the one experiment or a two-tailed unpaired t-test when only one compound was tested (Prism 4, Graphpad Software).

### Results

As seen in Tables 9 and 10, neither compounds (1) nor (3) significantly inhibited the ear swelling induced by the application of the inflammogens. However, compound (1) demonstrated a trend towards inhibiting the inflammation due to both inflammogens.

TABLE 9
Change in ear thickness in response to the application of AA

| | Change in ear | % Change | |
|---|---|---|---|
| | thickness (mean ± | compared with | |
| Compound | SD, ×0.01 mm) | vehicle | Significance |
| (1) | 24.1 ± 0.1 | −7.0 | NS |
| vehicle | 25.9 ± 1.1 | | |
| (3) | 22.2 ± 1.9 | −5.9 | NS |
| vehicle | 20.9 ± 2.0 | | |

TABLE 10
Change in ear thickness in response to the application of PMA

| | Change in ear | % Change | |
|---|---|---|---|
| | thickness (mean ± | compared with | |
| Compound | SD, ×0.01 mm) | vehicle | Significance |
| (1) | 28.9 ± 3.071 | −12.3 | NS |
| vehicle | 31.8 ± 3.05 | | |
| (3) | 37.9 ± 2.6 | 0 | NS |
| vehicle | 37.7 ± 3.1 | | |

### Example 3

### Antioxidant Activity

Inflammatory processes are linked to oxidative cell damage, and there is ample evidence of the anti-inflammatory effects of antioxidants. Little is known about the underlying molecular mechanisms, although one hypothesis is that they inhibit the production of proinflammatory cytokines and adhesion molecules. Compounds (1) and (3) have been demonstrated in a number of assays to have very robust antioxidant activity.

### 3.1 Effect on Free Radical Scavenging

### Methods

The antioxidant (free radical trapping) activity of test compounds was assessed using the stable free radical compound 2,2-diphenyl-1-picrylhydrazyl (DPPH). A stock solution of DPPH was prepared at a concentration of 0.1 mM in ethanol and mixed for 10 minutes prior to use. Compound (1) was reacted with DPPH for 20 minutes, after which time the absorbance at 517 nm was determined. The change in absorbance at 517 nm was compared to a reagent blank (DPPH with ethanol alone). The IC₅₀ value was estimated as the concentration of the test compound that caused a 0.6 change in absorbance (with 1.2 absorbance units representing total scavenging of the DPPH radical).

### Results

As shown in Table 11, both compounds (1) and (3) displayed potent antioxidant activity.

TABLE 11
Free radical scavenging ability of test compounds - EC₅₀ (μM)

| Compound | EC₅₀ (μM) |
|---|---|
| (1) | 18.8 |
| (2) | >100 |
| (3) | 13.6 |

### 3.2 Effect on Inhibiting the Oxidation of Low Density Lipoprotein (LDL)

Oxidized low density lipoprotein (LDL) is pro-inflammatory, it can cause endothelial dysfunction and it readily accumulates within the arterial wall (Rosenson 2004). Oxidized lipoproteins are thought to provoke a number of changes in cell functions that promote atherogenesis, via an inflammatory response. Therefore, inhibition of the oxidation of LDL can be anti-atherogenic, anti-inflammatory and cardioprotective.

### Methods

Blood was collected by venipuncture and plasma separated by centrifugation. LDL was then isolated from plasma using a 4-step sodium chloride density gradient and ultracentrifuged at 200,000 g for 20 hours at 4° C. The collect LDL was purified by passage through gel filtration PD10 column to remove excess salt and EDTA, and stored in the dark at 4° C. to prevent auto-oxidation and used with two weeks of isolation. The LDL cholesterol content was measure using a standard enzymatic method and protein concentration determined by the Lowry method using BSA as the standard.

On the day of each experiment a 2 mL aliquot of LDL was passed through a second PD10 column and diluted with chelex treated PBS (100 mM) to give a standard protein concentration of 0.1 mg/mL, ie final concentration per reaction. Oxidation reactions were initiated by the addition of freshly prepared Cu²⁺ solution, such that the final concentration of CuSO₄ was 5 μM. For inhibition studies, LDL was pre-treated with either compound (1) or compound (3), at final concentrations of 0.1, 1.0, 10 and 100 μM, for 2 minutes at room temperature prior to the addition of copper solution and subsequently incubated at 37° C. The extent of lipoprotein oxidation was determined by measuring the formation of lipid-peroxides on aliquots removed every 30-minute over a 3-hour period. Peroxides were determined at each time point by the ferrous oxidation-xylenol orange (FOX) assay using standard hydrogen peroxide curve (5 to 200 μM). Compound (1) was examined in at least two separate experiments performed on separate days.

The non-specific binding of compound (1) to Cu⁺⁺ was also examined in duplicate on different days. A stock solution of test compounds was prepared in DMSO at a concentration of 5 mM. The UV/Vis absorption spectra of was then determined between 200 and 800 nm after dilution of test compounds to 25 μM in phosphate buffer (10 mM, pH 7.2, chelex treated). Interactions of the compounds with Cu(II) were determined by scanning a second overlaying absorption spectrum over 200 to 800 nm, in which 25 μM CuSO₄ solution was added to a fresh 25 μM solution of test compounds and mixed for 20 seconds.

### Results

Reference to Tables 12 to 15 below demonstrates that both compounds (1) and (3) were very active at inhibiting the oxidation of LDL. The LDL oxidation lag period was approximately 60 minutes and maximum oxidation was achieved by 120-180 minutes. The ability of compounds (1) and (3) to inhibit LDL oxidation increased with increasing concentration from 0.01 to 10 μM. The concentration at which 50% of the oxidation was inhibited, the IC₅₀ was calculated as 0.58 μM for compound (1) and 0.5 μM for compound (3).

There were no significant shifts to absorbance bands of test compounds with Cu²+ at a 1:1 molar ratio. There was a very small and consistent increase in the absorbance bands of compound (1) with Cu²⁺ compared to the compounds alone. From these results it can be concluded that compound (1) did not interact with Cu²⁺. This also indicates that the underlying mechanisms of inhibition of LDL oxidation are most likely not due to a direct interaction of Cu²⁺ ions with the test compounds.

TABLE 12
Raw data for time-course lipid peroxide
formation in the absence of compound (1)

| Time | | | LDL 0.1 mg/ml + |
|---|---|---|---|
| (min) | LDL 0.1 mg/ml | Cu⁺⁺ 5 μM | Cu⁺⁺ 5 μM |
| 0 | 9.18 ± 1.32 | 3.76 ± 1.54 | 12.59 ± 1.73 |
| 30 | 9.37 ± 0.50 | 3.35 ± 0.43 | 15.71 ± 0.94 |
| 60 | 8.90 ± 0.93 | 3.29 ± 1.11 | 31.90 ± 1.58 |
| 90 | 8.84 ± 0.60 | 3.26 ± 1.32 | 112.12 ± 3.98 |
| 120 | 8.90 ± 0.29 | 2.53 ± 0.81 | 176.28 ± 6.31 |
| 180 | 9.60 ± 0.07 | 1.70 ± 3.67 | 193.10 ± 17.24 |

TABLE 13
Raw data for time-course lipid peroxide
formation in the presence of compound (1)

| | | | | | Compound (1) |
|---|---|---|---|---|---|
| Time | LDL 0.1 mg/ml + compound (1) + Cu⁺⁺ 5 μM | | | | 100 μM + |
| (min) | 0.1 μM | 1.0 μM | 10 μM | 100 μM | Cu⁺⁺ 5 μM |
| 0 | 11.54 ± 1.96 | 11.31 ± 1.95 | 11.54 ± 1.96 | 13.83 ± 1.77 | 10.13 ± 2.82 |
| 30 | 14.50 ± 0.28 | 12.83 ± 2.28 | 16.77 ± 2.55 | 29.71 ± 6.85 | 32.09 ± 11.15 |
| 60 | 26.86 ± 0.65 | 13.11 ± 2.70 | 19.56 ± 4.55 | 39.57 ± 10.67 | 46.21 ± 20.68 |
| 90 | 89.43 ± 17.03 | 14.78 ± 1.06 | 20.84 ± 5.30 | 78.85 ± 33.54 | 58.19 ± 28.98 |
| 120 | 161.78 ± 1.44 | 20.39 ± 2.95 | 22.80 ± 3.39 | 54.24 ± 9.58 | 69.46 ± 35.33 |
| 180 | 193.20 ± 25.05 | 102.30 ± 37.30 | 20.30 ± 4.97 | 62.50 ± 19.58 | 76.60 ± 40.17 |

TABLE 14
Raw data for time-course lipid peroxide
formation in the absence of compound (3)

| Time | | | LDL 0.1 mg/ml + |
|---|---|---|---|
| (min) | LDL 0.1 mg/ml | Cu 5 μM | Cu⁺⁺ 5 μM |
| 0 | 0.36 ± 4.62 | 1.29 ± 3.24 | 6.344 ± 2.74 |
| 30 | −0.51 ± 2.09 | −2.21 ± 4.52 | 1.716 ± 10.9 |
| 60 | 3.69 ± 6.39 | −2.66 ± 1.88 | 27.809 ± 5.01 |
| 90 | 4.16 ± 5.17 | −2.35 ± 1.19 | 105.735 ± 7.35 |
| 120 | 4.77 ± 6.21 | −3.74 ± 1.81 | 142.144 ± 8.29 |
| 150 | 5.43 ± 9.28 | −6.86 ± 4.88 | 147.264 ± 13.29 |

TABLE 15
Raw data for time-course lipid peroxide formation in the presence of compound (3)

| | | | | | Compound (3) |
|---|---|---|---|---|---|
| Time | LDL 0.1 mg/ml + compound (3) + Cu⁺⁺ 5 μM | | | | 10 μM + |
| (min) | 0.01 μM | 0.1 μM | 1.0 μM | 10 μM | Cu⁺⁺ 5 μM |
| 0 | 5.55 ± 0.22 | 5.62 ± 0.85 | 7.38 ± 2.69 | 1.62 ± 4.14 | 2.36 ± 5.25 |
| 30 | 2.85 ± 2.80 | 3.24 ± 1.93 | −2.09 ± 9.59 | 2.59 ± 2.96 | −3.75 ± 3.02 |
| 60 | 25.92 ± 13.53 | 18.89 ± 7.84 | 7.21 ± 4.53 | 5.65 ± 2.55 | −1.38 ± 2.64 |
| 90 | 88.97 ± 3.03 | 73.79 ± 1.40 | 7.13 ± 3.76 | 4.40 ± 1.13 | −0.82 ± 2.31 |
| 120 | 131.94 ± 4.81 | 138.28 ± 2.75 | 11.69 ± 7.30 | 3.57 ± 2.23 | −2.48 ± 2.55 |
| 150 | 137.86 ± 26.03 | 155.66 ± 21.85 | 79.63 ± 27.82 | 4.15 ± 2.87 | −6.22 ± 6.83 |

### 3.3 Effect on Peroxyl Radical-Induced Red Blood Cell (RBC) Lysis

### Methods

Freshly collected heparinised venous blood (10 ml, on ice) was aliquotted into 1.8 ml sterile eppendorf tubes and centrifuged for 10 minutes at 2600 rpm at 4° C. Plasma and buffy coat layers were removed (approximately 900 μl) and packed red blood cells (RBC) were then washed by the addition of 900 μl of sterile, ice cold PBS. This washing procedure was repeated twice. Packed RBC were resuspended by the addition of 900 μl of ice-cold, sterile PBS (and termed RBC stock). RBC stocks were stored at 4° C. for a maximum of three days. All working suspensions of RBC were prepared fresh daily by diluting 200 μl of RBC stock into 10 ml of ice-cold, sterile PBS and 50 μl added to each well.

Stocks of AAPH were freshly prepared for individual experiments as follows. AAPH (1.22 gm) was dissolved in 7.5 ml of PBS to yield a 4× stock at 600 mM and 50 μl aliquots (final concentration of 150 mM) were then added to each well to initiate the lysis assay. Stock solutions of test compounds (40 mM in 100% DMSO) were diluted in sterile PBS to yield final concentrations of 100, 30 and 10 μM per well. Appropriate controls were included in each experiment. Dilutions were adjusted to give final DMSO concentrations in each well of 0.25%. Peroxyl-induced RBC lysis assays were performed in 96-flat bottom well microtitre plates with a total volume of 200 μl per well. Turbidity of RBC suspensions were monitored using a Tecan microplate reader at 690 nm (37° C.) with gentle vortexing. Assays were performed in quadruplicate and readings were taken every 5 minutes over 5 hours. RBC lysis curves were constructed by plotting absorbance (mean of 4 readings) against time. Time to half-lysis was calculated by taking the highest absorbance reading (no lysis) and the lowest absorbance reading (maximum lysis). The sum of these two readings divided by two gave the absorbance at half-lysis. Simple regression analysis was used to calculate the time at which half-lysis absorbance occurred.

### Results

As shown below in Table 16, compound (1) demonstrated antioxidant activity by delaying the AAPH-induced time to half-lysis of red blood cells.

TABLE 16
Time taken to reach half-lysis following incubation
with test compounds at 1 μM (min)

| Compound | time (min) |
|---|---|
| vehicle | 40.0 |
| Compound (1) | 154.6 |
| Compound (3) | 88.6 |

### Example 4

### Immunomodulating Activity

Rheumatoid arthritis is a chronic, inflammatory, multisystem, autoimmune disorder that usually manifests with polyarthritis. The pathogenesis involves a T-cell mediated ‘attack’ on the synovium. Inflammatory bowel disease (IBD) is considered to be an inappropriate immune response in genetically susceptible individuals as the result of a complex interaction among environmental factors, microbial factors, and the intestinal immune system. Both diseases are often treated with a combination of anti-inflammatory and immunosuppressive therapies. Selected compounds of the invention were therefore tested in order to determine whether they have immunosuppressive activity in addition to anti-inflammatory activity.

### Methods

Male Skh-1:HR1 (hairless) mice, approximately six weeks old were killed by cervical dislocation. Single cell suspensions were made from the spleen and erythrocytes were lysed in buffer (0.14M NH4Cl, 17 mM Tris, pH 7.2). The remaining splenocytes were cultured in RPMI-1640 (Gibco) supplemented with 10% (v:v) FBS, 200 mM L-glutamine, penicillin/streptomycin and 50 mM 2-mercaptoethanol. Splenocytes were added to quadruplicate wells containing either concanavalin A (ConA, Sigma-Aldrich—0.4 μg/well), LPS (Sigma-Aldrich—1 μg/well) or no mitogen, as well 10 μM of test compound in DMSO. Samples were analysed after a 3 day incubation at 37° C. in 5% CO2 in air. Methylthiazoletetrazolium (MTT) is bioreduced by viable cells into a coloured formazan product that is soluble in DMSO. Thus the quantity of formazan product is directly proportional to the number of living cells in culture, and can be measured using a spectrophotometer at 570 nm. MTT was added to each well, incubated for a further 4 hrs and then colour developed with 0.04N HCl in isopropanol. Culture supernatants were stored after collection at −80° C. and both T and B cells analysed by ELISA (BD Biosciences) for IFN-γ (a Th-1 cytokine) and for T cells alone, IL-6 (a Th-2 cytokine).

### Results

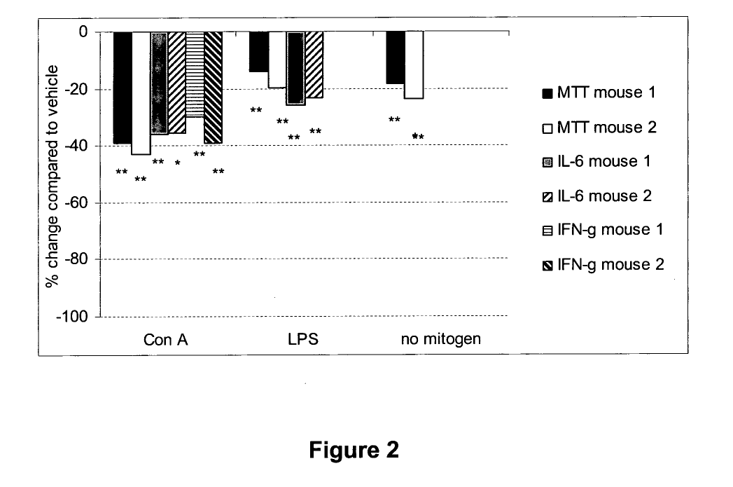

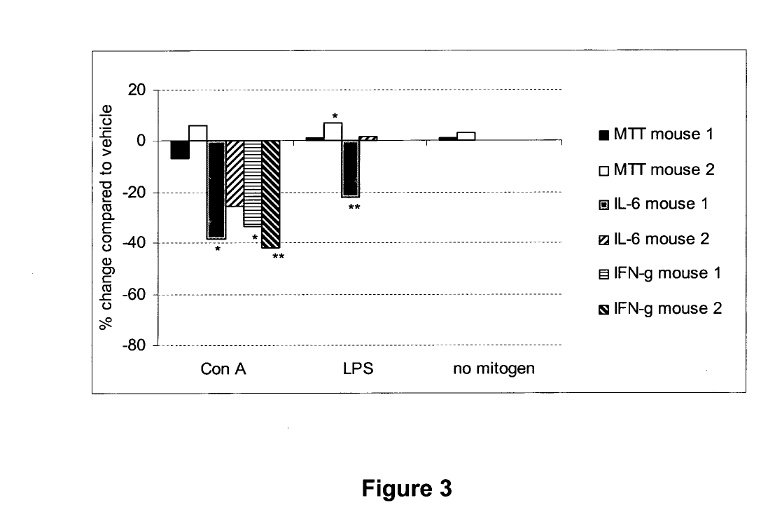

Compounds (1) and (3) were examined in two individual mice each. Compound (1) was markedly and significantly immunosuppressive to T cells and to a lesser extent, B cells. This effect was further evidenced by a concomitant reduction on the synthesis of INF-γ and IL-6 into the supernatant (see FIGS. 2 and 3).

In contrast, compound (3) had little effect on cell numbers, but did decrease the cytokines produced by T cells in particular.

Both compounds (1) and (3) therefore appear to have immunosuppressive attributes.

### Example 5

### Cardioprotective Activity

As discussed above, anti-inflammatory activity via COX inhibition is associated with an increase in the occurrence of adverse cardiovascular events. This can be mediated by the inhibition of prostacyclin (PGI₂) or the tendency for selective COX-2 inhibition to provide an increase in PGH₂, the substrate for the pro-thrombotic thromboxane A₂.

### 5.1 Effect on Prostacyclin Production

PGI₂ is the main COX product of endothelial cells, produced from PGH₂ by the action of the enzyme prostacyclin synthase.

Its actions of vasodilation and inhibition of platelet aggregation can be considered anti-thrombotic. PGI₂ additionally protects from cardiovascular disease by pleiotropic effects on vascular smooth muscle cells (VSMC). Genetic deletion of the prostacyclin receptor in mice reduced the development of atherosclerosis, intimal hyperplasia and restenosis, possibly via PGI₂ inhibition of VSMC proliferation and migration. Its production is inhibited indirectly by NSAIDs, via inhibition of COX, and it is this effect that contributes to the increase in adverse cardiovascular events associated with all NSAIDs. Therefore, a desirable cardioprotective attribute of an anti-inflammatory agent would be lack of inhibition of endothelial PGI₂ synthesis.

### Methods

Cultured human umbilical vein endothelial cells (HUVECs—Vascular Biology Laboratory, Hanson Institute, Adelaide SA) were removed harvested in 0.25% trypsin-EDTA. After quenching and further washing in RPMI-10% FCS, the cells were resuspended in fresh medium at 1-1.5×10⁵ cells per ml and plated out in gelatin coated wells at 2 ml per well. After incubation overnight in 5% CO₂ at 37° C., the medium was refreshed and the cells returned to the incubator and allowed to equilibrate for approximately 2 h. Test compounds at concentrations of 0, 1, 10 or 100 μM were added to the cells, and 30 minutes later, stimulated by interleukin-1β (IL-1β-10 μl of a 2 ng/ml solution). After overnight incubation at 37° C., supernatants were collected by centrifugation at 2000 rpm for 5 min and stored at −20° C. Production of prostacyclin after overnight incubation was measured by radioimmunoassay (RIA). Because PGI₂ is labile in aqueous medium, the stable hydrolysis product 6-keto PGF_{1α}, was measured as a surrogate marker. Results are expressed as mean+SEM, n=3. Differences between means were analysed by one-way ANOVA followed by Tukey's test for multiple comparisons. Differences between means were regarded as significant when p<0.05.

An effect on cell growth was observed microscopically at 100 μM but not at the lower doses. Normal HUVECs were predominantly epithelioid but included some spindle shaped cells. Cells were mainly healthy and viable, as indicated by their translucent appearance and adherence to the culture dish. Floating cells were presumed non-viable. Confluent growth in the central region of the wells resulted in the classic “cobblestone” appearance. In non-confluent areas, adherent cells had a “stretched” appearance.

### Results

Reference to Table 17 below shows that compound (1) had no effect at 1 μM, but significant inhibition occurred at the higher concentrations of 10 μM and 100 μM, whereas Compound (3) had no effect at any concentration tested.

TABLE 17
Effect of test compounds on 6-keto PGF1α synthesis
in IL-1β stimulated HUVECs

| | PGF_{1α} (pg/ml) | | | |
|---|---|---|---|---|
| Compound | 0 μM | 1 μM | 10 μM | 100 μM |
| (1) | 0.885 ± 0.038 | 0.667 ± 0.088 | 0.584 ± 0.059 * | 0.277 ± 0.07 * |
| (3) | 0.486 ± 0.071 | 0.353 ± 0.045 | 0.335 ± 0.039 | 0.308 ± 0.065 |

* significantly different from control (0 μM) (P < 0.05)

Microscopically, cells treated with compound (1) and compound (3) appeared healthy. The therapeutic implication is that at physiologically relevant concentrations, compounds (1) and (3) would have minimal if any pro-thrombic activity.

### 5.2 Effect on Thromboxane and Prostoglandin Synthesis

TXA₂ produced in activated platelets by TXS has prothrombotic properties by stimulating platelet aggregation and vasoconstriction. Inhibiting TXS selectively would therefore be anti-thrombotic. This might be evidenced by a shunting of substrate to enable an increase in the synthesis of PGE₂. The effect of test compounds was examined in human monocytes and the murine macrophage cell line, RAW 264.7. COX inhibitory activity would be evidenced by substantial inhibition of both PGE₂ and TXB₂.

### Method 1—Human Monocytes

U937 cells were thawed and resuspended in RPMI and 10% FCS at 2×10⁵ cells per ml. The cells were incubated in 5% CO₂ at 37° C. and expanded in growing culture to at least 6.4×10⁷ total cells. The cells were then resuspended in fresh medium and cultured with 5 μM retinoic acid (RA) at 2×10⁵ cells per ml for a further 3 days (72 h). RA treated cells were washed 2× in serum-free RPMI and resuspended in serum-free medium at 5×10⁶ cells per ml. The cells were aliquotted into Teflon tubes at 1 ml per tube. Working stock solutions of compounds (1) and (3) were prepared at 0.1 mM, 1 mM and 10 mM as described above. For each working dilution, 10 μl was added to 1 ml cells to achieve a final concentration of 0 (DMSO alone), 1, 10, and 100 μM for each test compound. Cells were incubated in triplicate with each concentration of test compound for 15 min at 37° C. After 15 min pre-incubation, each 1 ml tube of cells received 5 μl of a 100 mM solution of the calcium ionophore A23187 (to achieve 0.5 μM A23187). Incubation at 37° C. was continued for a further 30 min. After incubation, supernatants were collected by centrifugation at 2000 rpm for 10 min and stored at −20° C. until required for assay.

### Results

Reference to Tables 18 and 19 below shows that at the highest dose of 100 μM, compounds (1) and (3) inhibited the synthesis of both eicosanoids, which was probably due to cytotoxicity. However, at the lower concentrations, compounds (1) and (3) tended to increase the synthesis of PGE₂, with little effect on TXB₂. Thus, it can be reasoned that they have no COX inhibitory activity.

TABLE 18
Effect of test compounds on PGE₂ synthesis in RA-stimulated U937 cells

| | PGE₂ (pg) | | | |
|---|---|---|---|---|
| Compound | 0 μM | 1 μM | 10 μM | 100 μM |
| (1) | 1.226 ± 0.087 | 1.485 ± 0.175 | 1.58 ± 0.145 | 0.881 ± 0.116 |
| (3) | 0.736 ± 0.064 | 1.025 ± 0.114 | 1.271 ± 0.212 | 1.39 ± 0.227 |

TABLE 19
Effect of test compounds on TXA₂ synthesis in RA-stimulated U937 cells

| | TXB₂ (pg) | | | |
|---|---|---|---|---|
| Compound | 0 μM | 1 μM | 10 μM | 100 μM |
| (1) | 39.287 ± 6.005 | 35.833 ± 1.866 | 41.697 ± 2.275 | 21.708 ± 5.439 |
| (3) | 16.019 ± 2.701 | 18.434 ± 0.367 | 26.276 ± 9.76 | 12.668 ± 3.506 |

### Method 2—Murine Macrophage Cell Line, RAW 264.7

The mouse macrophage cell line RAW 264.7 was cultured in DMEM supplemented with foetal calf serum (FCS), 2 mM glutamine and 50 U/ml penicillin/streptomycin. Cells were treated with either test compounds (in 0.025% DMSO) or vehicle alone, and added one hour before 50 ng/ml LPS. After incubation for 16 hrs, culture media was collected for PGE₂ or TXB₂ measurement by ELISA (Cayman Chemical), and TNFα measurement using an ELISA (Becton Dickinson).

### Results

As shown in Tables 20 and 21 below, at 10 μM, compound (1) reduced the synthesis of PGE₂. However, this effect may have been influenced by toxicity in RAW 264.7, where the IC₅₀ is 53.9±1.204. Otherwise, as with the human monocytic cell line, there was no evidence of COX inhibition by compound (1). Compounds (1) and (2) have little effect on the viability of RAW 264.7 cells, and so it can be concluded that they exhibited evidence of weak COX inhibitory activity only, in this assay.

TABLE 20
Effect of test compounds on PGE₂ synthesis in RAW 264.7

| | PGE₂ (pg/ml) | | |
|---|---|---|---|
| Compound | 0 μM | 1 μM | 10 μM |
| (1) | 2973.71 ± 406.9 | 2601.37 ± 153.53 | 847.96 ± 144.83 |
| (2) | 4522.7 ± 116.43 | 3180.37 ± 127.29 | 2070.69 ± 168.81 |
| (3) | 3416.45 ± 286.45 | 2890.02 ± 320.91 | 2031.43 ± 261.59 |

TABLE 21
Effect of test compounds on TXA₂ synthesis in RAW 264.7

| | TXB₂ (pg/ml) | | |
|---|---|---|---|
| Compound | 0 μm | 1 μM | 10 μM |
| (1) | 1712.39 ± 691.24 | 1333.17 ± 121.78 | 1149.14 ± 480.27 |
| (2) | 15.29 ± 1.79 | 16.71 ± 3.43 | 17.58 ± 7.99 |
| (3) | 348.0 ± 115.35 | 374.8 ± 132.29 | 287.04 ± 75.58 |

There was little effect on the synthesis of TNFα as shown in Table 22 below.

TABLE 22
Effect of test compounds on TNFα synthesis in RAW 264.7

| | TNFα (pg/ml) | | |
|---|---|---|---|
| Compound | 0 μM | 1 μM | 10 μM |
| (1) | 50463.99 ± 6293.44 | 40002.31 ± 694.8 | 41958.94 ± 837.04 |
| (2) | 31749.96 ± 1346.64 | 35085.88 ± 2273.16 | 46065.55 ± 1234.32 |
| (3) | 30518.23 ± 2413.44 | 26677.52 ± 815.64 | 45429.44 ± 1630.68 |

### 5.3 Vasodilatory Activity in the Art Aortic Ring Assay

The vasodilatory capacity of the compounds of formula (1) was examined ex situ using the rat aortic ring assay. The addition of noradrenaline to the test bath causes the rings to contract, and if that vasoconstriction is inhibited by a test agent i.e. it antagonises the effect of noradrenaline, it suggests that that agent may have vasodilatory activity.

### Methods

Male Sprague-Dawley rats (250±50 g) were euthanased with 80% CO₂ and 20% O₂. The thoracic aorta was excised and quickly mounted in organ-baths as described (Chin-Dusting et al. 2001). Full concentration-contractile curves were obtained to noradrenaline (0.1 nM-10 mM) with and without test compounds delivered at a concentration of 1 μg/ml. Experiments were repeated in n=5 different rings from 5 different animals. Only one compound at any one concentration was tested on any one ring from any one animal. Sigmoidal dose response curves were fitted for the data and the logEC₅₀ calculated (Prism 4, GraphPad Software). The difference in these values between the presence and absence of test compound was calculated using a two-tailed paired t test. The effects of β-oestrodiol and vehicle alone were examined as a positive and negative control respectively.

### Results

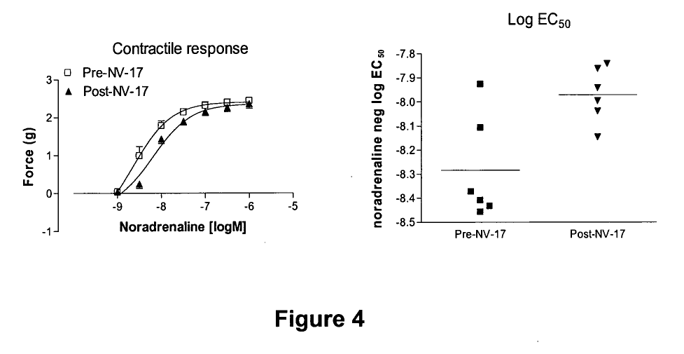

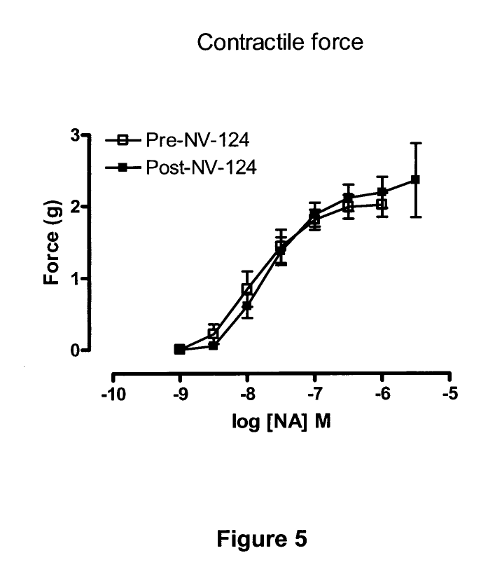

Compound (1) (p=0.045) significantly inhibited the contractile response (logEC₅₀) of the aortic ring to noradrenaline compared with vehicle alone by 23% (see FIGS. 4 and 5).

### Example 6

### Pharmacokinetics

### Methods

The pharmacokinetic (PK) profiles of compounds (1) and (3) were examined following oral administration in PEG 400/PBS 1:1 at a dose of 25 mg/kg. For each experiment, three animals were allocated per time point (15 min, 30 min, 60 min, 90 min, 4 hr and 24 hr). Mice were killed using cervical dislocation and serum collected via cardiac puncture. Faeces and urine, where available, were also collected. Samples were stored at −80° C. and analysed by LC-MS in-house. The limit of detection was 20 ng/ml.

### Results

As seen in Table 23 below, whilst neither the [max] or AUC of compound (1), for free or total levels was particularly high in circulation, the amount excreted in urine was relatively high, suggesting that it was well absorbed but rapidly excreted. The rate of conjugation is relatively low at 33%. There was also a suggestion of a biphasic peak, with the second at 90 min post-administration, which suggests the possibility of some enterohepatic circulation.

In Table 23 (and Table 24), “AUC” represents the area under the serum concentration versus time curve, expressed as μM*hour/L. This number assesses absorption and clearance in a relative way. The higher the number, the more compound has been absorbed and the longer it has remained in circulation. “AUC_{free}” refers to the area under the curve for unconjugated or free analogue, whereas “AUCₜₒₜₐₗ” refers to the area under the curve for the free and conjugated analogue combined.

“[max]” refers to the maximum concentration observed in serum. These measurements also give some understanding of how well a compound is absorbed. However, it does not take into consideration how rapidly it is either conjugated and/or excreted, so that a compound may have a very low maximum concentration, yet still be well absorbed.

The ratio of the AUC_{free} to the AUCₜₒₜₐₗ gives a relative measure of how much of the administered compound i.e. ‘free’ remains either in circulation compared to how much of the compound is conjugated. Therefore, if the ratio is relatively high, it suggests that much of the compound remains unconjugated, whereas if the ratio is relatively low, it suggests that conjugation (and thus perhaps urinary excretion) occurs rapidly.

“t_{1/2}” is the half life, i.e. the time taken for the serum concentration to fall by half. The elimination of a drug is usually an exponential (logarithmic) process, so that a constant proportion is eliminated per unit time. These data were generated by non-linear regression, using an equation for one phase exponential decay. The first refers to the t_{1/2} of the unconjugated analogue and the second the t_{1/2} of the total (free plus conjugated) analogue.

TABLE 23
PK profile of compound (1) following a single oral dose in mice

Compound
(1)

| | serum | urine |
|---|---|---|
| AUC_{free} | 7.6 | 558.2 |
| [max]_{free} (μM, mean ± SD) | 0.13 ± 0.1 | 5.6 ± 6.7 |
| AUCₜₒₜₐₗ | 23.2 | 58978 |
| [max]ₜₒₜₐₗ (mean ± SD) | 0.9 ± 0.5 | 188.9 ± 70.2 |
| AUC_{free}/AUCₜₒₜₐₗ | 33% | — |
| t_{1/2} (free - total) | 9 - 19 mins | — |

The data in Table 24 below shows that compound (3) would appear to be less well absorbed than compound (1), at least when delivered in this vehicle to mice—although its AUCₜₒₜₐₗ is higher than that of compound (1), the maximum concentration observed in circulation is much lower. Whilst the rate of conjugation appears higher, the half life may be longer than that of compound (1).

TABLE 24
PK profile of compound (3) following a single oral dose in mice

Compound
(3)

| | serum | urine |
|---|---|---|
| AUC_{free} | 6.5 | 2043 |
| [max]_{free} (μM, mean ± SD) | 0.03 ± 0.01 | 3.9 ± 2.4 |
| AUCₜₒₜₐₗ | 54.4 | 3326 |
| [max]ₜₒₜₐₗ (mean ± SD) | 0.3 ± 0.1 | 5.5 ± 3.4 |
| AUC_{free}/AUCₜₒₜₐₗ | 12% | — |
| t_{1/2} (free - total) | 28 - 11.5 mins | — |

### Example 7

### Toxicity in Normal Cells

### Methods

Human neonatal fibroblast foreskin (NFF—a gift from Dr. Peter Parsons, Queensland Institute of Medical Research) or rabbit kidney (RK-13—a gift from Prof. Miller Whalley, Macquarie University) cells were seeded into 96 well plates and cultured in RPMI supplemented with 10% FCS (CSL, Australia), penicillin (100 U/ml), streptomycin (100 mg/ml), L-glutamine (2 mM) and sodium bicarbonate (1.2 g/L) at 37° C. and 5% CO₂ for 24 hours until cells had attached and entered log phase of growth. Test compounds were added in serial two-fold dilutions from 150 μM in triplicate and incubated for a further 5 days. MTT was then added to each well, incubated for 3 hours at 37° C., after which the medium was tipped off. Following the addition of DMSO, the absorbance for each well was read on a plate reader. The assays were repeated at least twice.

### Results

As shown below in Table 25 below, compounds (1), (2) and (3) were without toxicity in RKs at the highest concentration tested. Compound (1) demonstrated mild toxicity to NFFs, whereas compounds (2) and (3) can be considered “non-toxic”.

TABLE 25
Effect of the test compounds on normal cell viability (IC₅₀ - μM)

| Compound | NFF | RK |
|---|---|---|
| (1) | 64.1 ± 9.6 | 150.0 ± 0 |
| (2) | 145.3 ± 8.1 | 150.0 ± 0 |
| (3) | 111.2 ± 67.1 | 150.0 ± 0 |

### Example 8

### Activity in Cancer Cell Lines

### Methods

The human colorectal cell line HT-29 (HTB-38™), human prostate lines PC-3 (CRL-1435™) and DU-145 (HTB-81™), and the human melanoma line SK-Mel-28 (HTB-72™) were cultured in RPMI 1640 medium (Gibco, Cat#21870-076).

The human prostate cell line LNCaP Clone FGC (CRL-1740™), human leukemic cell line CCRF-CEM™ (CCL-119™) human colorectal adenocarcinoma cell line HCT-15 (CCL-225™) and the human lung cancer cell lines NCI-H23 (CRL-5800™) and NCI-H460 (HTB-127™) were cultured in RPMI 1640, supplemented to contain 10 mM HEPES (Sigma, Cat#H0887), 4.5 g/L Glucose (Sigma, Cat#G8769) and 1 mM Sodium Pyruvate (Sigma, Cat#S8636).

The human melanoma cell line MM200 was obtained as a gift from Prof. Peter Hersey (University of Newcastle) and cultured in Dulbecco's Modified Eagle's Medium (DMEM) (Gibco, Cat#11960-069).

The human melanoma cell line MM96L were obtained as gift from Professor Peter Parsons (Queensland Institute of Medical Research) and cultured in RPMI 1640.

The human ovarian cancer cell lines A2780 and CP70 were obtained as gifts from Dr. Gil Mor (Yale University). A2780 was cultured in RPMI 1640 medium. CP70 was cultured in DMEM/Hams F-12 1:1 (Gibco, Cat#11320-082) supplemented with 10 mM HEPES, 1× non essential amino acids (Sigma, Cat#M7145), 5.0 g/L sodium bicarbonate (Sigma, Cat#55761), and 1 mM sodium pyruvate.

The breast cancer cell line MDA-MB-468 (HTB-132™) was cultured in DMEM/Hams F-12 1:1. The human pancreatic cancer cell line, HPAC (CRL-2119™) was routinely cultured in DMEM/Hams F-12 1:1 and supplemented with 15 mM HEPES, 0.002 mg/ml insulin (Sigma, Cat#I9278), 0.005 mg/ml transferrin (Sigma, Cat#T8158), 40 ng/ml hydrocortisone (Sigma, Cat#H0135) and 10 ng/ml epidermal growth factor (Sigma, Cat#E4269).

The human Glioma cell line Hs 683 (HTB-138™) was cultured in DMEM.

All cultures with the exception of HPAC and CP70 were supplemented with 2 mM L-Glutamine (Gibco, Cat#25030)

All cultures were supplemented with 10% FBS (Gibco, Cat#10099-158), 5000 U/ml penicillin and 5 mg/ml streptomycin (Gibco, Cat#15070), and cultured at 37° C. in a humidified atmosphere of 5% CO₂.

All cell lines were purchased from ATCC (Maryland, USA) except where noted.

IC₅₀ values were determined for each cell line. Cells were seeded in 96-well plates at an appropriate cell density as determined from growth kinetics analysis and cultured for 5 days in the absence and presence of the test compounds. Cell proliferation was assessed after the addition of 20 μl of 3-4,5dimethylthiazol-2,5-diphenyl tetrazolium bromide (MTT, 2.5 mg/ml in PBS, Sigma) for 3-4 hrs at 37° C. according to manufacturer's instructions. IC₅₀ values were calculated from semi-log plots of % of control proliferation on the y-axis against log dose on the x-axis.

### Results

As shown in Table 26 below, compound (1) demonstrated activity (ie IC₅₀<20 μM) in a number of cancer cell lines. Compounds (2) and (3) were less active.

TABLE 26
Effect of the test compounds on inhibition of cancer cell lines

| | | Analogue (IC₅₀ μM) | | |
|---|---|---|---|---|
| | | Geometric Mean */Standard Deviation | | |
| | | (Log-Normal Distribution) | | |
| Indication | | Compound (1) | Compound (2) | Compound (3) |
| Ovarian | A2780 | 9.94 */2.04 | 25.35 */1.00 | 38.71 */1.00 |
| Leukaemia | CCRF-CEM | 150.00 */1.00 | 63.40 */2.37 | 94.49 */1.59 |
| Ovarian | CP70 | 8.57 */1.05 | 105.42 */1.65 | 130.81 */1.21 |
| Prostate | DU145 | 5.54 */1.00 | — | — |
| Colorectal | HCT-15 | — | 62.82 */1.00 | 134.58 */1.00 |
| Pancreatic | HPAC | 18.59 */1.00 | 76.78 */1.95 | 110.85 */1.11 |
| Colorectal | HT29 | 40.54 */1.00 | 134.38 */1.15 | 139.48 */1.11 |
| Glioma | HTB-138 | 7.97 */1.05 | 150.00 */1.00 | 150.00 */1.00 |
| Prostate | LNCaP | 60.22 */1.33 | 150.00 */1.00 | 150.00 */1.00 |
| Pancreatic | MDA-MB-468 | 3.99 */1.00 | 76.41 */1.26 | 104.99 */1.38 |
| Melanoma | MM200 | 14.33 */1.00 | 68.25 */1.33 | 54.66 */1.41 |
| Melanoma | MM96L | — | 52.87 */1.00 | 46.77 */1.00 |
| Lung | NCI-H23 | — | 150.00 */1.00 | 150.00 */1.00 |
| Lung | NCI-H460 | 4.44 */1.01 | 66.47 */1.68 | — |
| Prostate | PC3 | 14.04 */2.15 | 150.00 */1.00 | 91.09 */2.02 |
| Melanoma | SKMel-28 | — | 96.06 */1.00 | 110.16 */1.00 |

### REFERENCES

Boyum, A. (1986). “Separation of leukocytes from blood and bone marrow.” Scand J Clin Invest 21: 77-89.
Caughey, G. E., L. G. Cleland, et al. (2001). “Up-regulation of endothelial cyclooxygenase-2 and prostanoid synthesis by platelets. Role of thromboxane A2.” Journal of Biological Chemistry 276(41): 37839-45.
Chin-Dusting, J. P., L. J. Fisher, et al. (2001). “The vascular activity of some isoflavone metabolites: implications for a cardioprotective role.” British Journal of Pharmacology. 133(4): 595-605.
Fosslien, E. (2005). “Cardiovascular Complications of Non-Steroidal Anti-Inflammatory Drugs.” Ann Clin Lab Sci 35(4): 347-385.
Gilroy, D. W., A. Tomlinson, et al. (1998). “Differential effects of inhibition of isoforms of cyclooxygenase (COX-1, COX-2) in chronic inflammation. [comment].” Inflammation Research. 47(2): 79-85.
Kobayashi, T., Y. Tahara, et al. (2004). “Roles of thromboxane A2 and prostacyclin in the development of atherosclerosis in apoE-deficient mice.” J. Clin. Invest. 114(6): 784-794.
Mita, H., Y. Yui, et al. (1988). “Isocratic determination of arachidonic acid 5-lipoxygenase products in human neutrophils by high-performance liquid chromatography.” J Chromatogr 430(2): 299-308.
Rosenson, R. S. (2004). “Statins in atherosclerosis: lipid-lowering agents with antioxidant capabilities.” Atherosclerosis 173(1): 1-12.

1. A compound of the general formula (I): 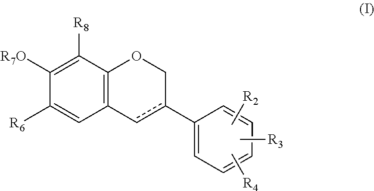
wherein:
R₂, R₃ and R₄ are independently selected from the group consisting of: hydrogen, hydroxy, OR₉, OC(O)R₉, OSi(R₁₀)₃, C₁-C₁₀ alkyl, C₃-C₇ cycloalkyl, amino, aminoalkyl, aryl, arylalkyl, alkylaryl, thiol, COOH, alkylthio, nitro, cyano, halo, C₂-C₆ alkenyl, C₂-C₆ alkynyl and heteroaryl,
R₆ is R₁₁(R₁₂)N(CH₂)ₙ—,
R₇ is selected from the group consisting of: hydrogen, R₉, C(O)R₉, Si(R₁₀)₃ and C₃-C₇ cycloalkyl,
R₈ is selected from the group consisting of: hydrogen, C₁-C₁₀ alkyl, C₃-C₇ cycloalkyl, aryl, arylalkyl, nitro, cyano and halo,
R₉ is selected from the group consisting of C₁-C₁₀ alkyl, haloalkyl, aryl, arylalkyl and alkylaryl,
R₁₀ is independently selected from the group consisting of C₁-C₁₀ alkyl and aryl,
R₁₁ and R₁₂ are independently selected from the group consisting of: hydrogen, C₁-C₁₀alkyl and —Y—CO₂R₁₃, or R₁₁ and R₁₂ together with the nitrogen to which they are attached form a heterocyclic ring comprising 5, 6 or 7 ring members, the heterocyclic ring being optionally substituted with one or more substituents selected from the group consisting of: C₁-C₁₀ alkyl, C₂-C₆ alkenyl, C₂-C₆ alkynyl, COOH, COOR₁₀, halo, nitro, cyano and aryl,
R₁₃ is selected from the group consisting of: hydrogen, C₃-C₇ cycloalkyl, C₁-C₁₀ alkyl, C₂-C₆-alkenyl and C₂-C₆ alkynyl,
Y is a hydrocarbon chain having between 1 and 15 carbon atoms which may optionally be interrupted by one or more oxygen, nitrogen or sulfur atoms,
n is an integer between 1 and 4,
the drawing [symbol] represents either a single bond or a double bond, and salts thereof.

2. The compound of claim 1, wherein R₂, R₃ and R₄ are independently selected from the group consisting of: hydrogen, C₁-C₁₀ alkyl, halo, hydroxy, OR₉, OC(O)R₉ and OSi(R₁₀)₃.

3. The compound of claim 1, wherein R₂, R₃ and R₄ are independently selected from the group consisting of: hydrogen, hydroxy and OR₉.

4. The compound of claim 1, wherein R₇ is selected from the group consisting of hydrogen, C(O)R₉ and C₁-C₁₀ alkyl.

5. The compound of claim 1, wherein R₈ is selected from the group consisting of: hydrogen, C₁-C₁₀ alkyl and halo.

6. The compound of claim 1, wherein R₉ is selected from the group consisting of: C₁-C₁₀ alkyl, haloalkyl and aryl.

7. The compound of claim 1, wherein R₁₀ is C₁-C₁₀ alkyl.

8. The compound of claim 1, wherein R₁₁ and R₁₂ are independently selected from the group consisting of: —Y—CO₂R₁₃, hydrogen and C₁-C₁₀ alkyl, or R₁₁ and R₁₂ together with the nitrogen to which they are attached form a heterocyclic ring comprising 5 or 6 ring members, the heterocyclic ring being optionally substituted with one or more substituents selected from the group consisting of: C₁-C₁₀ alkyl, COOH, COOR₁₀ and halo.

9. The compound of claim 8, wherein R₁₁ and R₁₂ are independently selected from the group consisting of: —Y—CO₂R₁₃, hydrogen and C₁-C₆ alkyl, or R₁₁ and R₁₂ together with the nitrogen to which they are attached form a heterocyclic ring comprising 5 or 6 ring members, the heterocyclic ring being optionally substituted with one or more substituents selected from the group consisting of: C₁-C₁₀ alkyl, COOH and halo.

10. The compound of claim 1, wherein Y is a hydrocarbon chain having between 1 and 6 carbon atoms.

11. The compound of claim 1, wherein R₁₃ is C₁-C₆ alkyl.

12. The compound of claim 1, wherein n is 1, 2 or 3.

13. The compound of claim 1, wherein at least one of R₂, R₃ and R₄ is hydroxy.

14. A compound of claim 1, which is compound (1), (2), (3), (4), (5), (6), (7) or (8).

15. A pharmaceutical composition comprising a compound of the formula (I) according to claim 1, or a pharmaceutically acceptable salt thereof, and a pharmaceutically acceptable carrier, diluent and/or excipient.

16. A method for the prevention and/or treatment of inflammation and/or an inflammatory disease or disorder in a subject in need thereof, said method comprising administration to the subject of a therapeutically effective amount of a compound of the formula (I) according to claim 1, or a pharmaceutically acceptable salt thereof.

17. Use of a compound of the formula (I) according to claim 1, or a pharmaceutically acceptable salt thereof, as an antioxidant.

18. A method for modulation of the immune system in a subject, said method comprising administration to the subject of a therapeutically effective amount of a compound of the formula (I) according to claim 1, or a pharmaceutically acceptable salt thereof.

19. A method for inhibiting the proliferation of cells, said method comprising contacting the cells with a compound of the formula (I) according to claim 1, or a pharmaceutically acceptable salt thereof.

20. A method for the prevention and/or treatment of cancer in a subject in need thereof, said method comprising administration to the subject of a therapeutically effective amount of a compound of the formula (I) according to claim 1 or a pharmaceutically acceptable salt thereof.

21. The method of claim 20, wherein the cancer is selected from the group consisting of: ovarian cancer, leukaemia, prostate cancer, colorectal cancer, pancreatic cancer, glioma, melanoma and lung cancer.

22. A method for the prevention and/or treatment of cardiovascular disease in a subject in need thereof, said method comprising administration to the subject of a therapeutically effective amount of a compound of the formula (I) according to claim 1 or a pharmaceutically acceptable salt thereof.
